(19) United States
(12) Patent Application Publication
(10) Pub. No.: US 2012/0258128 A1
(43) Pub. Date: Oct. 11, 2012

(54) Infection Factor Assay

(75) Inventors: Andrea J. Dowling, Penryn Cornwall (GB); Richard H. Ffrench-Constant, Penryn Cornwall (GB)

(73) Assignee: UNIVERSITY OF EXETER, Exeter Devon (GB)

(21) Appl. No.: 13/438,453

(22) Filed: Apr. 3, 2012

Related U.S. Application Data
(60) Provisional application No. 61/473,395, filed on Apr. 8, 2011.

(51) Int. Cl. A61K 39/02; A61K 39/00; A61P 31/04; C40B 30/06; C07K 14/195; A61P 37/04; C07K 1/00; C12N 15/31

(52) U.S. Cl. 424/190.1; 530/350; 424/184.1; 536/23.7; 506/10; 424/234.1

(57) ABSTRACT

There is provided a method of selecting an anti-macrophage micro-organism comprising an anti-macrophage factor, the method comprising the steps of:
a) obtaining an assay micro-organism and preparing a sample thereof;
b) lysing the assay micro-organism cells contained in the sample from (a) to form a lysate fluid;
c) contacting a sample of macrophage cells with the lysate fluid from step (b);
d) determining the macrophage cell viability and comparing the viability to the viability of macrophage cells in a control macrophage sample; and
e) selecting the micro-organism as an anti-macrophage micro-organism if the viability is reduced by at least 10%.

The anti-macrophage factors identifiable by methods according to the invention can be used in the formulation of vaccines and other therapies against disorders caused by the anti-macrophage micro-organism.

### CROSS-REFERENCE TO RELATED APPLICATIONS

This application claims the benefit of U.S. provisional application No. 61/473,395 filed Apr. 8, 2011, which is hereby incorporated by reference in its entirety.

### FIELD OF INVENTION

The invention relates to the identification of factors within micro-organisms which contribute to their anti-macrophage activity and to compositions and vaccines developed using such factors, as well as to methods of prophylaxis and treatment of disorders caused by anti-macrophage micro-organisms.

### INCORPORATION OF SEQUENCE LISTING

The entire contents of a computer readable form and a computer software generated sequence listing entitled 439199_SequenceListing_ST25.txt, which is 99 kilobytes in size and was created on Mar. 30, 2012, are herein incorporated by reference.

### BACKGROUND

Development of vaccines against pathogenic organisms is an important strategy to prevent and treat diseases caused by such organisms. Typically, a vaccine contains an agent resembling or derived from the infectious organism and is used to stimulate an immune response in the treated individual. For example, a vaccine may be the organism itself in killed or attenuated form. Alternatively, a vaccine may comprise a component of the organism, such as a protein subunit fragment of an organism outer coat, or an inactivated version of a toxic compound used by the organism to cause harm in the host body. Development of any such vaccine composition requires knowledge and understanding of the infection pathway and/or lifecycle of the organism and such information is not well understood for all organisms.

For example, the Gram-negative bacterium Burkholderia pseudomallei is a serious environmental pathogen of man and the causative agent of the often fatal disease melioidosis. Disease occurs following exposure to contaminated water or soil, usually through cuts in the skin or via inhalation, but the underlying mechanisms of pathogenicity of B. pseudomallei to humans remain poorly understood (Adler et al. (2009) FEMS Microbiol. Rev. 33:1079-1099). B. pseudomallei is endemic to S.E. Asia and N. Australia where infections are associated with both antibiotic resistance and high mortality rates (−50%). The high rates of infection and subsequent patient mortality make B. pseudomallei a high priority for research and vaccine development, as no effective vaccine currently exists.

During the establishment of successful infection B. pseudomallei adheres to, survives and replicates within host epithelial cells and macrophages by somehow interfering with the cellular mechanisms which would otherwise destroy them. Known bacterial factors affecting the interaction with host cells include the bacterial capsule, and effectors delivered by the type III and type VI secretion systems (T3SS and T6SS) (Galyov et al., Annu Rev Microbiol. (2010) 64:495-517). Once inside the macrophage the pathogen induces macrophage cell fusion leading to the formation of so called Multi-Nucleated Giant Cells or MNGCs, a process key to both intracellular replication and bacterial persistence but one for which the molecular basis is obscure (Kespichayawattana et al. (2000) Infect. Immun. 68:5377-5384). Once intracellular replication of the pathogen has reached a critical point the bacteria induce host cell death (again by an unknown mechanism) and subsequently escape host cells to establish secondary infections (Adler et al. (2009) FEMS Microbiol. Rev. 33:1079-1099).

Different Burkholderia strains show a wide range of different interactions with human macrophages, ranging from no effect, to host cell apoptosis and caspase-1-dependent lysis. This range of different responses to macrophages suggests that the complement of anti-macrophage virulence factors encoded by the genome of different strains may differ dramatically and may also indicate potential functional redundancy amongst such factors. Importantly, conventional genomic analysis has failed to identify homologues of known toxins in B. pseudomallei (Holden et al. (2004) Proc. Natl. Acad. Sci. USA 101:14240-14245. For example, whilst a cytolethal exotoxin has been identified in the culture filtrate of B. pseudomallei, the toxin remains to be identified and the encoding gene to be characterised (Haase et al. (1997) J. Med. Microbiol. 46:557-563).

There is, therefore, a need to develop new screens for potential virulence factors in Burkholderia and other pathogenic organisms, and for therapeutic and prophylactic treatments against organisms in which the infection and lifecycle pathways are poorly or incompletely defined.

### SUMMARY OF INVENTION

According to a first aspect of the invention there is provided a method of selecting an anti-macrophage micro-organism comprising an anti-macrophage factor, the method comprising the steps of:

a) obtaining an assay micro-organism and preparing a sample thereof;
b) lysing the assay micro-organism cells contained in the sample from (a) to form a lysate fluid;
c) contacting a sample of macrophage cells with the lysate fluid from step (b);
d) determining the macrophage cell viability and comparing the viability to the viability of macrophage cells in a control macrophage sample; and
e) selecting the micro-organism as an anti-macrophage micro-organism if the viability is reduced by at least 10%, for example, by at least 20%, 30%, 40%, 50%, 60%, 70%, 80%, 90% or 100%, for example if the macrophage cells are killed.

The method enables the identification of anti-macrophage factors which are not identifiable using conventional genomic analysis techniques. An “anti-macrophage factor” is a protein or other compound produced by, or associated with, the micro-organism which has a negative impact on macrophage viability as determined, for example, using the XTT assay described below and in Scudiero et al. (Cancer Res. (1988) 48:4827-4833). In this assay, cleavage of XTT (a yellow tetrazolium salt) to formazan (a soluble orange dye) is measured, the cleavage occurring due to enzymic reactions in metabolically active mitochondria. Alternative methods to determine macrophage viability can involve measuring functional cell processes such as membrane impermeability to certain dyes, or measurement of enzyme activity. The “negative impact” may be direct, for example by being toxic to the macrophages, or indirect, for example by involvement in synthesis of macrophage toxin or in delivery of a toxin to the macrophage.

A “control macrophage sample” is a sample equivalent to the sample contacted with the lysate in step (c), the control sample not being so contacted (or contacted with an equivalent fluid not comprising the assay micro-organism and/or the lysate thereof). A macrophage sample may be, for example, a confluent layer of BALB/c monocyte macrophages as described herein, or may be another type of macrophage sample as will be understood by the skilled person.

In general, the term “micro-organism”, as used anywhere in this specification, encompasses bacteria, yeasts, viruses, archaea and protists. The anti-macrophage micro-organism and/or the assay micro-organism may be a bacterium, for example an E. coli or Bacillus bacterium.

The step of preparing a sample of the assay micro-organism may include growing a micro-organism sample on a suitable surface such as an agar plate, or in a suitable liquid medium. The step may include sub-steps involving a period of growth on a surface followed by growth in a liquid medium, or vice versa. The skilled person is readily able to determine suitable surfaces and/or media for facilitating growth of a particular micro-organism such as a bacterium and will also be able to determine incubation conditions and time periods needed for sample preparation to provide a sample suitable for use in the method. By way of example, a bacterium might be grown on a microplate containing a Luria medium for 24 hours at 37° C., agitated at 350 rpm.

The lysing step (b) may include adding lysozyme to the sample from (a) as a means of lysing the micro-organism cells. This may be combined or replaced by a freeze-thaw method, also to break open the cells. Other methods of lysing the cells may be utilised, as will be understood by the skilled person.

The step of obtaining an assay micro-organism may comprise introducing genomic nucleic acid such as DNA from a test micro-organism into an expression micro-organism and using the expression micro-organism as the assay micro-organism. The expression micro-organism may be known to be not an anti-macrophage micro-organism, so that any effect on the macrophages can be attributed to the inclusion of the test micro-organism genomic nucleic acid. This provides the advantage that the genome of a dangerous micro-organism such as a micro-organism in Biohazard levels 2, 3 or 4 (as categorized by the US Centers for Disease Control) can be transferred to a non-hazardous micro-organism for study. Even when the non-hazardous micro-organism does not include the cellular apparatus to enable secretion of certain anti-macrophage factors, the method enables the identification of these factors, since a cell lysis step is included. Therefore, the anti-macrophage factors of a hazardous micro-organism can be rapidly and fully studied without the restrictions, hindrances and dangers associated with carrying out research on or using the pathogenic test micro-organism itself.

The test micro-organism may be a bacterium, for example a Biohazard level 2, 3 or 4 bacterium such as (but in no way restricted to) Burkholderia pseudomallei. Alternatively or additionally, the assay micro-organism and expression micro-organism may be a bacterium, for example, an E. coli or Bacillus bacterium. Yeasts are also especially contemplated for use as assay/expression micro-organisms in the method of the invention.

A related aspect of the invention provides a method of identifying a gene encoding an anti-macrophage factor comprising the method of the invention and further comprising the additional steps of:

(aa) before step (a) above, preparing a genomic library of the test micro-organism genome in an expression micro-organism and obtaining expression micro-organism clones, each of which comprises an assay micro-organism; and
(f) after step (e) above, determining the nucleotide sequence of the test micro-organism nucleic acid in the selected assay micro-organism; and
(g) using the nucleotide sequence to identify an equivalent sequence in a test micro-organism gene and selecting the gene as encoding an anti-macrophage factor.

This aspect of the invention enables the user to not only confirm that the genome of a test micro-organism contains a gene for an anti-macrophage factor, but enables the identification of the location or approximate location of the gene within the genome. The genomic library is most usefully, therefore, one for which the genome nucleic acid sequence is known. The use of such a genomic library, typically comprising portions of genomic nucleic acid (typically DNA but RNA is also contemplated) of around 10-100 kb (for example about 20 kb, about 30 kb, about 40 kb, about 50 kb or about 60 kb) in length, enables the user to determine a given region of the genome which is included and expressed by a particular clone, by way of comparison of the nucleic acid sequence in the clone with the known nucleic acid sequence of the library. The genomic nucleic acid may be contained in an expression vector such as, for example, a plasmid, bacterial artificial chromosome (BA), yeast artificial chromosome (YAC), fosmid or cosmid. The polynucleotide sequence may be operably linked to one or more expression sequences so as to enable expression of the gene(s) present in the nucleic acid.

Again, the test micro-organism may be a bacterium. Alternatively or additionally, each assay micro-organism and expression micro-organism may be a bacterium. When the expression and assay micro-organism is a bacterium, the genomic library may be a BAC library and/or a fosmid library; where the expression and assay micro-organism is a yeast, the genomic library may be a YAC library. The genomic library may also be a cosmid library.

In this aspect of the invention, the steps (a)-(f) above may be repeated with an assay micro-organism from at least two clones obtained in step (aa), in which case step (g) above is replaced with the steps of:

(fa) comparing the test micro-organism nucleotide sequence from each selected assay micro-organism with the sequence obtained from a different selected assay micro-organism to identify one or more regions of sequence overlap; and
(fb) using the nucleotide sequence in each region of overlap to identify an equivalent sequence in a test micro-organism gene and selecting the gene as encoding an anti-macrophage factor.

The region of sequence overlap may be of about 500 to about 40,000 nucleotides (0.5-40 kb), for example, at least about 0.5, 1, 2, 3, 4, 5, 10, 15, 20, 25, 30, 35 or 40 kilobases.

These methods may be used within a method of obtaining an isolated anti-macrophage factor, the method forming a second aspect of the invention. In addition to the methods described above, the method of obtaining an isolated anti-macrophage factor may further comprise expressing and isolating a protein encoded by the gene selected in step (g) or in step (fb).

The expressing may be carried out in a recombinant micro-organism such as a bacterium or yeast, or may be carried out using synthetic methods.

A third aspect of the invention provides a method of determining whether a test compound is an anti-macrophage factor, the method comprising introducing the compound into a micro-organism to form an assay micro-organism and carrying out the method of the first aspect of the invention. The test compound is identified as an anti-macrophage factor if the assay micro-organism is selected in step (e) as an anti-macrophage micro-organism.

The test compound may be a protein, in which case the assay micro-organism may be formed by introducing an expression system comprising a nucleic acid encoding the protein into the assay micro-organism. In one embodiment, the assay micro-organism is a bacterium, in which case the expression system may be a BAC, a fosmid or a cosmid. Where the assay micro-organism is a yeast, the expression system may be a YAC. Other expression systems are readily available to and adaptable by the skilled person, both for bacteria and for other micro-organisms.

In a fourth aspect of the invention, there is provided an anti-macrophage factor obtained and/or identified using the methods described above.

According to a fifth aspect of the invention, there is provided a vaccine composition comprising an attenuated or inactivated version of the anti-macrophage factor according to the fourth aspect of the invention and a pharmaceutically acceptable carrier. A pharmaceutically acceptable carrier may be any of the carriers well known in the art such as saline, buffer saline, dextrose, glycerol, etc. and may be adjusted to prepare a vaccine composition appropriate for the delivery method to be used, for example, an oral, nasal, intramuscular, subcutaneous or intravenous delivery method. Other delivery methods known in the art are contemplated.

According to a sixth aspect of the invention, there is provided a method of treatment or prevention of a disorder caused by an anti-macrophage micro-organism comprising obtaining and/or identifying an anti-macrophage factor as described above and administering a therapeutically or prophylactically effective amount of an attenuated or inactivated version of the factor, or of a vaccine as described above, to a subject in need thereof. The subject may be a mammal, for example a human subject. A disorder caused by an anti-macrophage micro-organism may be any disease or ailment, chronic or acute, associated with infection of a subject by or exposure of a subject to the micro-organism. Such associations are generally well understood.

According to a seventh aspect of the invention, there is provided an anti-macrophage factor polypeptide having an amino acid sequence comprising at least one of SEQ ID NO:1 (BPSL0590), SEQ ID NO:2 (BPSL0591), SEQ ID NO:3 (BPSS1381), SEQ ID NO:4 (BPSS1727), SEQ ID NO:5 (BPSS1728), SEQ ID NO:6 (BPS1266), SEQ ID NO:7 (BPSS1267), SEQ ID NO:8 (BPSS1268) or SEQ ID NO:9 (BPSS1269). SEQ ID NOs:6-9 together form the factor By1A. The factor polypeptide may also be any of those listed in Tables 1 and 2. These factors are derived and obtainable from the bacterium Burkholderia pseudomallei using methods as described herein in detail.

In a related aspect, there is provided a polynucleotide coding for an anti-macrophage factor polypeptide according to the seventh aspect of the invention. Using the standard genetic code, nucleic acids encoding the polypeptides may readily be conceived and manufactured by the skilled person. The nucleic acid may be DNA or RNA, and where it is a DNA molecule, it may for example comprise a cDNA or genomic DNA.

There is also provided a vaccine composition comprising an attenuated or inactivated version of one or more of the anti-macrophage factors according to the seventh aspect of the invention and a pharmaceutically acceptable carrier. There is also provided a vaccine composition comprising a polynucleotide encoding an attenuated or inactivated version of one or more of the anti-macrophage factors according to the seventh aspect of the invention and a pharmaceutically acceptable carrier.

The term “an attenuated or inactivated version of the factor” as used throughout this specification means that the naturally-occurring factor and/or the factor comprising one or more of SEQ ID NOs:1-9 and/or one or more factors listed in Tables 1 and 2 has been treated or altered in a way which reduces or eliminates its anti-macrophage activity. For example, this may be by alteration of the amino acid sequence if the factor is a protein, or by alteration of amendment of functional groups within the structure of the factor. Inactivation may also be carried out, for example, by heat treatment.

Vaccines and vaccine compositions, as described herein, may be attenuated or inactivated versions of the anti-macrophage factors of the invention which can be used to elicit an immune response in a subject to whom the vaccine or composition is administered. This means that the subject will be protected or partially protected from infection by an anti-macrophage micro-organism, typically a micro-organism in which the un-attenuated or non-inactivated version of the factor is found, so that exposure of the subject to the micro-organism does not result in their contracting an illness associated with the presence in a subject of the micro-organism. For example, the vaccine or composition may be useful to raise antibodies, capable of binding to an anti-macrophage factor and/or to a micro-organism comprising the factor, in a subject to whom the vaccine or composition is administered.

Where the factor is a protein, alteration of the amino acid sequence may be by altering the amino acid sequence from the base sequence from which it is derived in that one or more amino acids within the sequence are substituted for other amino acids, to form a anti-macrophage factor variant. Such a variant is one which is immunologically active, i.e., it will induce an antibody-mediated immune response so that antibodies may be produced by a cell to which the variant is exposed, those antibodies being capable of binding to a non-variant factor as described herein.

Amino acid substitutions may be regarded as “conservative” where an amino acid is replaced with a different amino acid with broadly similar properties. Non-conservative substitutions are where amino acids are replaced with amino acids of a different type.

By “conservative substitution” is meant the substitution of an amino acid by another amino acid of the same class, in which the classes are defined as follows:

| Class | Amino acid examples |
|---|---|
| Nonpolar: | A, V, L, I, P, M, F, W |
| Uncharged polar: | G, S, T, C, Y, N, Q |
| Acidic: | D, E |
| Basic: | K, R, H. |

In the present invention, conservative or non-conservative substitutions are possible provided that these do not result in a variant which is non-immunologically active, as defined above. For example, an immunologically active variant of an anti-macrophage factor according to the invention may have at least about 70% amino acid sequence identity at a global level, for example, at least about 75%, 80%, 85%, 90%, 95%, 96%, 97%, 98%, 99% or 100% sequence identity. The variant may also be a fragment of an anti-macrophage factor according to the invention, provided that the immunological activity is maintained. The level of immunogenicity of a non-variant anti-macrophage factor polypeptide may be measured by any standard means for measuring an immune response in a cell, for example, by determining the ability of the variant polypeptide to result in antibody generation by a cell to which the polypeptide is exposed, the antibody being capable of binding to an anti-macrophage factor according to the invention.

The invention encompasses nucleic acids encoding the anti-macrophage factor polypeptides of the invention and variants thereof. The term “variant” in relation to a nucleic acid sequence means any substitution of, variation of, modification of, replacement of, deletion of, or addition of one or more nucleic acid(s) from or to a polynucleotide sequence, providing the resultant polypeptide sequence encoded by the polynucleotide is an immunologically active variant of an anti-macrophage factor, as described herein. The term therefore includes allelic variants and also includes a polynucleotide (a “probe sequence”) which substantially hybridises to a polynucleotide coding for an anti-macrophage factor polypeptide according to the invention or an immunologically active variant thereof. Such hybridisation may occur at or between low and high stringency conditions. In general terms, low stringency conditions can be defined as hybridisation in which the washing step takes place in a 0.330-0.825 M NaCl buffer solution at a temperature of about 40-48° C. below the calculated or actual melting temperature (Tₘ) of the probe sequence (for example, about ambient laboratory temperature to about 55° C.), while high stringency conditions involve a wash in a 0.0165-0.0330 M NaCl buffer solution at a temperature of about 5-10° C. below the calculated or actual Tₘ of the probe sequence (for example, about 65° C.). The buffer solution may, for example, be SSC buffer (0.15M NaCl and 0.015M tri-sodium citrate), with the low stringency wash taking place in 3×SSC buffer and the high stringency wash taking place in 0.1×SSC buffer. Steps involved in hybridisation of nucleic acid sequences have been described for example in Sambrook et al. (1989; Molecular Cloning, Cold Spring Harbor Laboratory Press, Cold Spring Harbor).

Anti-macrophage factor polypeptides according to aspects of the invention may be prepared synthetically using conventional synthesizers. Alternatively, they may be produced using recombinant DNA technology or be isolated from natural sources followed by any chemical modification, if required. In these cases, a nucleic acid encoding the anti-macrophage factor is incorporated into a suitable expression vector, which is then used to transform a suitable host cell, such as a prokaryotic cell such as E. coli. The transformed host cells are cultured and the protein isolated therefrom. Vectors, cells and methods of this type form further aspects of the present invention.

Sequence identity between nucleotide and amino acid sequences can be determined by comparing an alignment of the sequences. When an equivalent position in the compared sequences is occupied by the same amino acid or base, then the molecules are identical at that position. Scoring an alignment as a percentage of identity is a function of the number of identical amino acids or bases at positions shared by the compared sequences. When comparing sequences, optimal alignments may require gaps to be introduced into one or more of the sequences, to take into consideration possible insertions and/or deletions in the sequences. Sequence comparison methods may employ gap penalties so that, for the same number of identical molecules in sequences being compared, a sequence alignment with as few gaps as possible, reflecting higher relatedness between the two compared sequences, will achieve a higher score than one with many gaps. Calculation of maximum percent identity involves the production of an optimal alignment, taking into consideration gap penalties.

Suitable computer programs for carrying out sequence comparisons are widely available in the commercial and public sector. Examples include the FASTA program (Pearson & Lipman, 1988, Proc. Natl. Acad. Sci. USA vol. 85 pp 2444-2448; Altschul et al., 1990, J. Mol. Biol. vol. 215 pp 403-410), ggsearch (part of the FASTA package) (Needleman & Wunsch, 1970, J. Mol. Biol. 48: 443-453), and the BLAST software. The latter is publicly available at http://blast.ncbi.nlm.nih.gov/Blast.cgi (accessed on 7 Apr. 2011) and sequence comparisons and percentage identities mentioned in this specification have been determined using this software. The FASTA program can be accessed publicly from the European Bioinformatics Institute (http://www.ebi.ac.uk/fasta) (accessed on 7 Apr. 2011). Typically, default parameters set by the computer programs should be used when comparing sequences. The default parameters may change depending on the type and length of sequences being compared. A comparison using the FASTA program may use default parameters of Ktup=2, Scoring matrix=Blosum50, gap=−10 and ext=−2. A comparison using the BLAST software may use the default parameters (scoring matrix=Blosum62, gap=11 and ext=1). As an alternative, the percentage sequence identity may be determined using the MatGAT v2.03 computer software, available from the website http://bitincka.com/ledion/matgat/ (accessed on 7 Apr. 2011). The parameters are set at Scoring Matrix=Blosum50, First Gap=16, Extending Gap=4 for DNA, and Scoring Matrix=Blosum62, First Gap=12, Extending Gap=2 for protein.

An eighth aspect of the invention provides a method of treatment or prevention of melioidosis comprising administering a therapeutically or prophylactically effective amount of an attenuated or inactivated version of a factor comprising one or more of the amino acid sequences SEQ ID NOs:1-9 and/or one or more polynucleotides encoding at least one of SEQ ID NOs:1-9 and/or a therapeutically or prophylactically effective combination of any of these and/or a vaccine composition comprising one or more of these to a subject in need thereof. The subject may be a mammal, for example a human subject.

Throughout the description and claims of this specification, the words “comprise” and “contain” and variations of the words, for example “comprising” and “comprises”, mean “including but not limited to” and do not exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Preferred features of each aspect of the invention may be as described in connection with any of the other aspects.

Other features of the present invention will become apparent from the following examples. Generally speaking, the invention extends to any novel one, or any novel combination, of the features disclosed in this specification (including the accompanying claims and drawings). Thus, features, integers, characteristics, compounds or chemical moieties described in conjunction with a particular aspect, embodiment or example of the invention are to be understood to be applicable to any other aspect, embodiment or example described herein, unless incompatible therewith.

Moreover, unless stated otherwise, any feature disclosed herein may be replaced by an alternative feature serving the same or a similar purpose.

### BRIEF DESCRIPTION OF FIGURES

Embodiments of the invention will now be described, by way of example only, with reference to FIGS. 1 and 2 in which:

FIG. 1 shows the distribution of features within positive loci on chromosome 1 and 2, displaying the number of CDSs for different functional classes found within anti-macrophage loci identified by the screen on both chromosomes; and

FIG. 2 shows the genomic organisation of four anti-macrophage loci isolated by the screen, representative of the main functional classes identified: BPSS1263-BPSS1269 encodes NRPS/PKS genes involved secondary metabolism including a putative efflux system and have homology to the Sy1A producing genes of P. syringae; BPSL0584-BPSL0591 encodes two hypothetical proteins (BPSL0590 and BPLS0591) with some homology to known toxins including SpvB of Salmonella enterica and Toxin complex components of Photorhabdus luminescens; a Phospholipase D-like protein is represented (BPSS1381), flanked by hypothetical proteins and core genome genes; a hemagglutinin/hemolysin related region BPSS1720-BPSS1728 has similarity to the filamentous hemagglutinin FHA of Bordetella pertussis.

### EXAMPLES

To perform the screen the inventors chose the strain B. pseudomallei K96246, a clinical isolate, whose genome, of two chromosomes, has been fully sequenced (Holden et al. (2004) Proc. Natl. Acad. Sci. USA 101:14240-14245). Chromosome 1 (4.07 Mb) represents 56% of the genome and contains a higher proportion of coding sequences (CDSs) than the smaller chromosome 2 (3.17 Mb). The CDSs on chromosome 1 are thought to be largely involved in housekeeping functions, such as metabolism, whereas those on chromosome 2 appear to encode accessory functions facilitating adaptation to atypical conditions, osmotic protection, secondary metabolism, iron acquisition and gene regulation. There are predicted to be at least 16 horizontally acquired genomic islands located in the B. pseudomallei genome which often contain genes encoding hypothetical virulence factors (Ho Sui et al. (2008) PLoS Pathog. 4:e1000178).

Libraries of recombinant E. coli each carrying end-sequenced B. pseudomallei genomic fragments (fosmids or Bacterial Artificial Chromosomes (BACs)) were used to identify loci encoding factors cytotoxic to the murine macrophage cell-line J774-2. The end-sequences of multiple positive clones recovered from the screens were aligned on to the sequenced genome in order to identify and confirm the precise configuration of the loci involved. Such a rapid and simple gain of function screen proves an extremely useful tool for dissecting pathogens displaying functional redundancy of multiple virulence factors and toxins. Such a multiplicity of bacterial virulence factors encoded within a single genome can frustrate attempts to dissect virulence via conventional mutagenesis.

For example, targeted knock-out of the toxin Mcf1 in Photorhabdus bacteria does not dramatically decrease anti-insect virulence due to the remaining copy of a second toxin encoding gene Mcf2 and a host of other remaining virulence factor encoding genes that remain unaffected (Waterfield et al. (2003) FEMS Microbiol. Lett. 229:265-270). Such ‘functional redundancy’ can, therefore, potentially mask the important role of specific gene candidates if other virulence factors compensate for the expected change in the resulting single mutant phenotype. Given the wealth of potential genes encoding putative virulence factors in the different genomes of B. pseudomallei, the inventors used this gain of function screening technique to reveal over 100 loci encoding anti-macrophage factors scattered across the two different chromosomes of this bacterial genome. Such analysis facilitates the identification and follow-up of effector proteins and small molecules that influence the potentially complex interaction between Burkholderia bacteria and host macrophages.

### Materials and Methods

### Genomic Library Construction

A combination genomic BAC and fosmid libraries, were used in these experiments. The BAC library was constructed in E. coli DH10B containing pBACe3.6 with an average B. pseudomallei DNA insert size of 20 kb originally employed for the Sanger genome sequencing project (Holden et al. (2004) Proc. Natl. Acad. Sci. USA 101:14240-14245). The Fosmid library was created using the CopyControl Fosmid Library Production Kit with E. coli EPI-300-T1^{R} (Epicentre) with an average insert size of 40 kb. BAC and fosmids libraries were arrayed into 96 well microplates to give ˜10× coverage of the genome. All clones in the libraries were then end-sequenced to facilitate location of their paired ends in the genome.

### Macrophage Toxicity Screening

Library plates were replicated into 96 well microplates containing 100 μl Luria Bertani medium plus 12.5 μg/ml chloramphenicol as the selective antibiotic for both the BAC and Fosmid library clones. Replicate library plates were grown for 24 h at 350 rpm, 37° C. and cultures were subsequently harvested by centrifugation at 4,000 rpm for 10 minutes. 80 μl of supernatant aspirated the remaining bacterial pellet and supernatant mixed thoroughly with 80 μl 1 mg/ml lysozyme in Phosphate Buffered Saline solution. Plates were then incubated at room temperature for a minimum of 1 h, followed by three freeze-thaw cycles before centrifugation at 3,000 rpm for 10 minutes. 80 μl of the crude lysates were removed and applied to 96 well plates containing confluent monolayers of the BALB/c monocyte macrophage cell line J774-2 (from The European Collection of Cell Cultures, ECACC) in Dulbecco's Modified Eagles Medium (DMEM) supplemented with 10% foetal bovine serum, 5% non-essential amino-acids and 5 μg/ml chloramphenicol. Crude lysates and macrophages were co-incubated for 24 h (37° C., 5% CO₂). Media on the macrophages was then replaced with phenol red-free DMEM containing an antibiotic cocktail: ampicillin 100 μg/ml, gentamicin 50 μg/ml, penicillin 100 U/ml, streptomycin 100 μg/ml, kanamycin 100 μg/ml and tetracycline 5 μg/ml and incubated with the macrophages for 2 h (37° C., 5% CO₂) to destroy live bacteria which would otherwise affect the readout of the cell viability assay. Macrophage cell viability was assessed using the XTT assay (Scudiero et al. (1988) Cancer Res. 48:4827-4833). Candidate positive BAC and Fosmid library clones were selected for their ability to reduce viability by 40% or more comparative to untreated cells.

### Candidate Identification

BAC and fosmid end sequences were aligned to the Burkholderia chromosomes using SSAHA2 version 2.5 (Ning & Mullikin (2001) Genome Res. 11:1725-1729) and the output exported in gff format. The alignments were manually checked to verify their integrity and to ensure the correct distance (10-20 kb) between mate-paired sequences. Once the gff had been edited it was uploaded to a custom Burkholderia GBrowse database, and the BAC and fosmid sequences were displayed as separate tracks in the GBrowse detail panel. This allows for the identification of ‘clusters’ of clones containing putative virulence factors. These positive ‘clusters’ form due to the multiplicity of genomic coverage within a fosmid library, for example, up to ten clones encoding an anti-macrophage toxin might be recovered from a library possessing 10× genomic coverage. The minimum region of genetic overlap within clusters was examined for candidate CDSs or operons using the annotated K96243 genome and BLASTX analysis (Stevens et al. (2002) Mol. Microbiol. 46:649-659). The distribution and location of positive loci on chromosome 1 and 2 were diagrammed using DNAPlotter (Carver et al. (2009) Bioinformatics 25:119-12)

### Characterisation of Cellular Phenotypes

Confluent monolayers of J774-2 macrophages on glass coverslips were treated with equivalent volumes of crude lysates (prepared as described above) from clones identified as containing regions of interest and co-incubated for 24 h. Coverslips were then washed in sterile 1×PBS before fixing with 4% paraformaldehyde (w/v) in PBS for 15 min. Coverslips were then washed in 1×PBS and immersed in a fresh solution of ammonium chloride in 1×PBS (13.3 mg/ml) for 15 minutes, at room temperature followed by washing in 1×PBS. Macrophages were permeabilized by covering with 0.2% Triton X-100 in 1×PBS for 15 minutes. Staining of the filamentous actin cytoskeleton was carried out with TRITC conjugated phalloidin (Sigma), at a 1/500 dilution in 1×PBS by inverting the coverslip onto a 60 μl drop of the staining solution and incubating at room temperature in the dark for 1 h. Following incubation the coverslips were washed 3×5 minutes in 1×PBS with the first wash containing 0.12 μg/μl Hoechst 33258 (Sigma) to stain the nuclei. A final wash by brief immersion 2× in distilled water is then made and coverslips mounted onto slides using ProLong Gold Antifade (Molecular Probes, Invitrogen) before visualization using fluorescence microscopy. For fraction screening of the Sy1A homolog a single colony of a BAC library clone containing the Sy1A region of homology was picked and grown for 24 h in LB plus 12.5 μg/ml chloramphenicol. Bacteria were harvested by centrifugation at 7,000 rpm for 5 minutes and the culture supernatant removed. Cell-free supernatant was prepared by filter-sterilizing with a 0.22 μm syringe-driven filter unit (Millipore). The cytosolic fraction was prepared by re-suspending the bacterial pellet in 1×PBS and sonicating the mixture. The resultant sonicated product was centrifuged at 10,000 rpm for 15 min. to remove cell debris from the cytosol preparation. Supernatant and cytosol fractions were applied to J774 macrophage monolayers at 1:5 (v/v) supernatant or cytosol: culture media and co-incubated for 24 h before fixing and staining as described.

### Results

### Loci Encoding Anti-Macrophage Factors are Distributed Over Both Chromosomes

The genomic libraries of B. pseudomallei K96243 in BAC and fosmid clones were screened for activity towards J774-2 macrophages. To identify and confirm anti-macrophage encoding loci in the B. pseudomallei genome the end-sequences from positive library clones (clones shown to reduce macrophage viability by >40%) were re-assembled onto the sequenced genome. Each locus was defined by the minimum region of genetic overlap formed by a cluster of two or more positive clones. Using these criteria, a total of 59 anti-macrophage encoding loci were identified on chromosome 1 and 54 on chromosome 2.

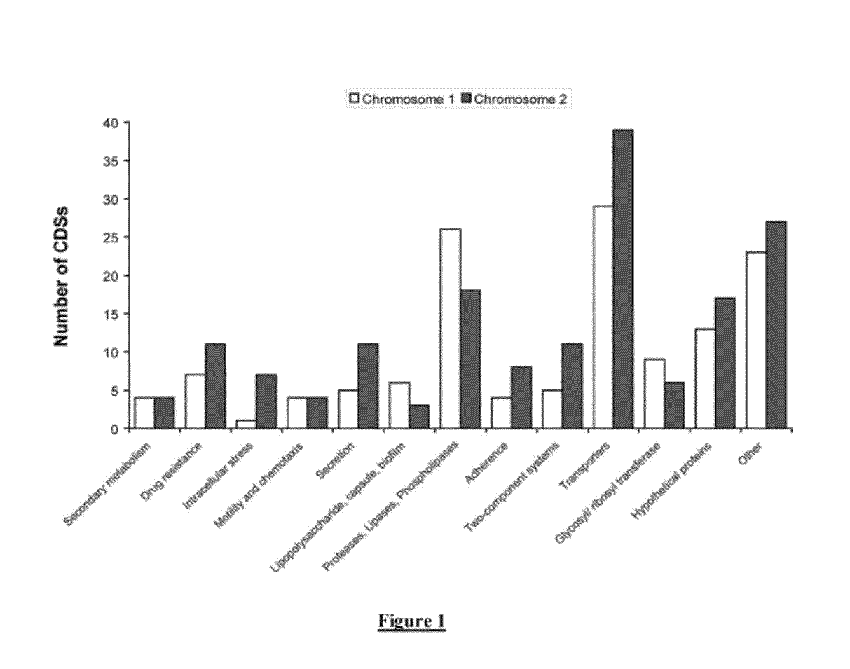

Several broad functional classes were repeatedly predicted for the anti-macrophage loci identified (FIG. 1). There are 14 predicted regions in K96243 containing putative secondary metabolite synthesis genes, 3 on chromosome 1 and 11 on chromosome 2. Six loci isolated by the screen contained clusters of genes predicted to encode non-ribosomal peptide synthetases (NRPSs) and polyketide synthases (PKSs). Genes involved in signal response and regulation (methyl-accepting chemotaxis proteins, two-component sensor systems, AraC and LysR family regulators) were also repeatedly detected. Numerous phage-related elements flank positive loci indicating that these may be mobile elements associated with insertion events and could be responsible for the potential transferal of virulence factors between B. pseudomallei strains. A number of genes which could promote the survival of B. pseudomallei in host cells were also identified in the regions of interest, including genes encoding factors responsible for resistance to drugs, heavy metals and reactive oxygen species (sodB and katB). Regions associated with anti-macrophage activity also include CDSs relating to adhesion (hemagglutinin, YD repeat proteins) and biofilm formation (lipopolysaccharide, acetolactate synthase). However, the largest predicted function categories associated with the anti-macrophage loci were either toxin/enzyme related (hemolysins, proteases, phospholipases) with 44 associated CDSs within loci, or proteins involved in transport/secretion (ABC transporters, efflux transporters, and proteins involved in type III and VI secretion) with 84 related CDSs within positive loci.

Several of the anti-macrophage loci encode putative toxins. For example, BPSS1993 encodes a metalloprotease A, termed MrpA, a 47 kDa protein negatively regulated by QS molecules, a putative hemolysin with homology to Bacillus cereus hemolysin III (BPSS0803) and LasA elastase from Pseudomonas aeruginosa (BPSL0624). One locus also contains a gene (BPSS0067) encoding phospholipase—PLC-3 a putative non-hemolytic phospholipase C with an N-terminal twin-arginine translocation (Tat pathway signal sequence) which was transcriptionally up-regulated in a hamster model of infection and increases the LD₅₀ of test animals dramatically (Tuanyok et al. (2006) Infect. Immun 74:5465-5476). Interestingly, the phospholipases PLC-1 (BPSL2403) and PLC-2 (BPSL0338) were not detected in this screen.

The genome of B. pseudomallei K96243 contains six gene clusters encoding putative T6SSs (Shalom et al. (2007) Microbiology 153: 2689-2699). Whilst, again, the host E. coli used in the library screens were not armed with any specific type III or type VI secretion systems, several positive loci encode putative VgrG-like T6SS-related proteins. Some VgrG effector proteins have been inferred to induce host cell toxicity by ADP-ribosylation of actin, as well as performing a role in formation of the secretion machinery itself (Pukatzki et al. (2009) Curr. Opin. Microbiol. 12:11-17; Suarez et al. (2010) J. Bacteriol. 192:155-168). B. pseudomallei T6SS-5 is induced upon exposure to macrophages and plays an important role in the intracellular survival of the pathogen (Shalom et al. (2007) Microbiology 153:2689-2699). However, a genomic clone containing the entire T6SS-5 cluster was not detected in the current study, presumably as either it did not fit entirely within one of the cloned library fragments or because other elements necessary for its production in the recombinant E. coli used were absent.

There are a total of 105 predicted functional ABC systems encoded within the genome of strain K96243 (Harland et al. (2007) BMC Genomics 8:83) and 22 of these were identified within the anti-macrophage encoding loci (14 on chromosome 1 and 8 on chromosome 2). Within this category of hits, the positive ABC transporter encoding loci include two class I export systems. One region of interest detected contains the LPS biosynthetic operon BPSL2672-BPSL2688 containing the class I wzt ABC transporter, confirming the potential role of LPS in interaction with macrophages (Matsuura et al. (1996) FEMS Microbiol. Lett. 137:79-83; Arjcharoen et al. (2007) Infect. Immun. 75:4298-4304) and suggesting that an active form of LPS can be correctly assembled by the host E. coli used for library construction. The second class I ABC system detected, BPSL3092-BPSL3094, is classified as involved in hemolysin export, although there are no identified hemolysins associated with this system in K96243 (Harland et al. (2007) BMC Genomics 8:83). However, this ABC system neighbours two putative peptidase encoding genes whose products may be exported. Further, the inventors noted that many of the predicted proteins from positive candidate regions are thought to encode outer membrane proteins, again consistent with their potential interaction with host macrophages and their presumptive display on the outer membrane of the recombinant E. coli used in the screen.

In summary, given that the host E. coli used in the screen (DH10B and EPI-300-T1^{R}) lack the specific secretion machinery (e.g. T3SS or T6SS) necessary for delivery of many types of known effectors, the clusters of anti-macrophage loci uncovered here appear to be able to reconstitute toxicity in the recombinant E. coli either using systems still present (e.g. type 1 secretion) or by using transporters (e.g. ABC) or synthetic machinery (e.g. liposaccharide biosynthesis) also encoded within the positive clones/clusters.

### Cellular Phenotypes of Anti-Macrophage Factors

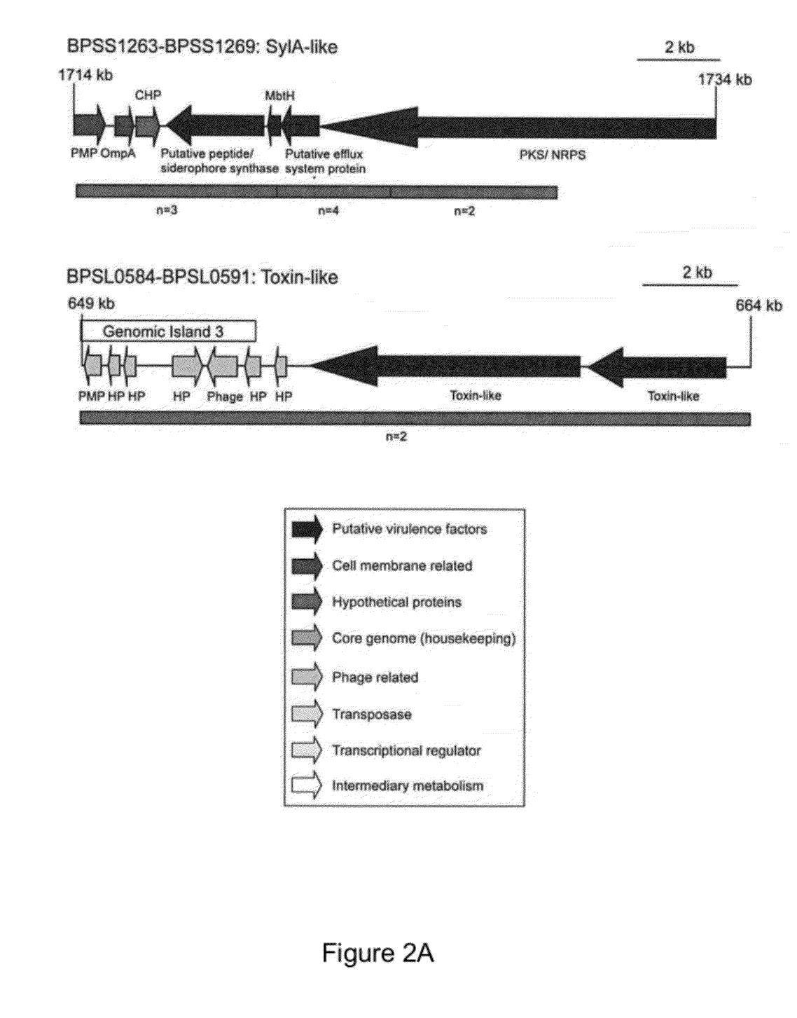

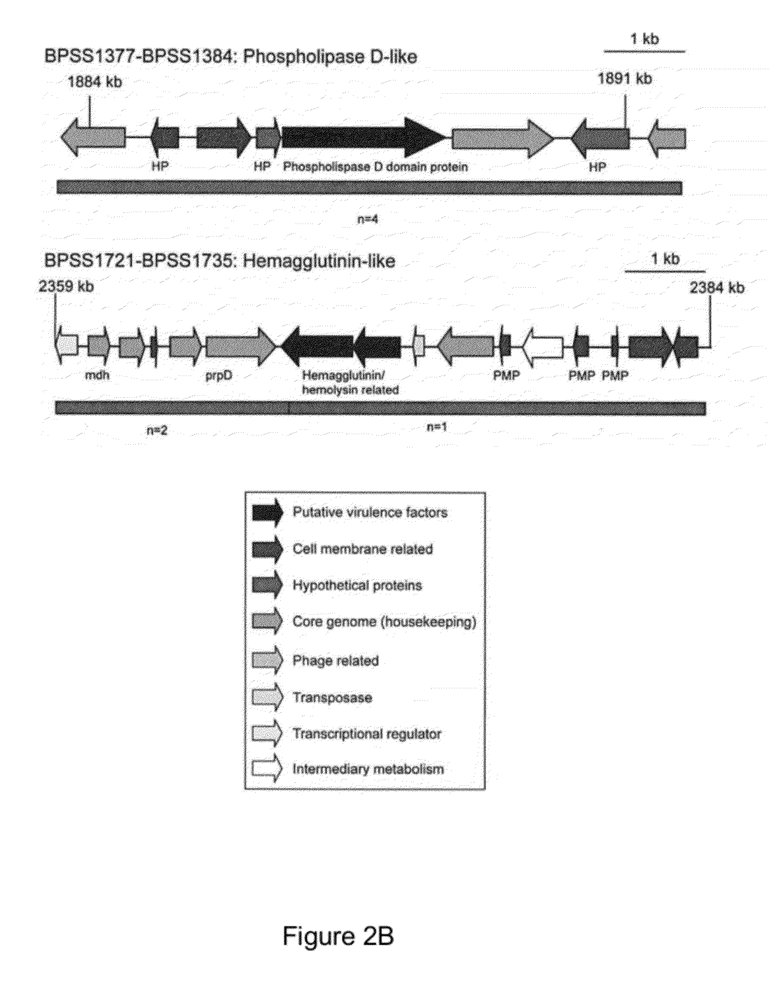

In order to begin to characterize the range of likely cellular phenotypes caused by this plethora of new candidate anti-macrophage factors, the inventors focused on the four positive clusters diagrammed in FIG. 2. These clusters carry CDSs encoding factors related to secondary metabolism (NRPS/PKS), candidate enzymes, bacterial adhesins and unclassified hypothetical proteins. The NRPS/PKS encoding region BPSS1263-BPSS1269 has homology to the Syringolin A (Sy1A) producing NRPS cluster from the plant pathogen Pseudomonas syringae (Groll et al. (2008) Nature 452:755-758). Sy1A is a proteome inhibitor and is cytotoxic to a number of mammalian cell types, including carcinoma derived cell lines and such compounds are, therefore, of interest as potential anticancer drugs (Groll et al. (2008) Nature 452:755-758; Coleman et al. (2006) Cell Prolif. 39:599-609). Macrophages treated with positive clones containing the Sy1A-like NRPS cluster show aberrations in nuclear morphology, with nuclei becoming enlarged and showing an irregular (here termed ‘corrugated’) perimeter. The cytoskeleton of the treated cells also becomes amorphous, punctate and collapses around the nucleus itself.

Thirty of the anti-macrophage loci contained ‘hypothetical proteins’ whose potential functions cannot be predicted from homology with known virulence factors. BPSL0590 and BPSL0591 are CDSs found in a positive locus on chromosome 1 which encode such hypothetical proteins. However, closer examination of protein predictions from these two CDSs does reveal some limited homology to known toxins from other bacterial pathogens, suggesting they may encode novel toxins. Position-specific-iterative blast (psi-BLAST) reveals that this putative membrane protein also has a central region similarity to the Rhs associated core sequence (1.98e-10) and to the middle N- and C-terminal domains of a Photorhabdus luminescens insecticidal Toxin complex (Tc) component protein, specifically TcdB N and C terminal regions (7.87e-36 and 7.90e-29 respectively). The N-terminal region of BPSL0590 has homology to the N-terminal of Salmonella enterica plasmid virulence associated protein SpvB (1.03e-09). The function of the N-terminus of SpvB remains unknown but shares sequence similarity to the N-terminal domain of P. luminescens toxin TcaC. The function of TcaC is largely unclear but is thought to act as a potentiator modifying other toxin components and it is required to reproduce full toxicity of the Toxin complexes via recombinant expression (Ffrench-Constant et al. (2005) Adv. Appl. Microbiol. 58C:169-183; Otto et al. (2000) Mol. Microbiol. 37:1106-1115). The catalytic domain of SpvB responsible for ADP-ribosylation of host cell actin is located at the C-terminus Depolymerization of host cell actin by SpvB causes destruction of cytoskeletal structure and cell death via apoptosis (Otto et al. (2000) Mol. Microbiol. 37:1106-1115; Libby et al. (2000) Cell. Microbiol. 2:49-58). Structural predictions for SpvB suggest that the C-terminus is linked to the N-terminus via a poly-proline region. This suggests that it could represent a class of modular toxins in which the active/enzymic region is linked to the N-terminus whose function remains undefined.

The neighbouring hypothetical protein BPSL0591 also displays predicted homology to a P. luminescens insecticidal Tc protein, specifically TccB. Macrophages treated with lysate from clones encompassing these toxin-like genes show both formation of multinucleated cells and apoptotic nuclei. These results suggest that this is an exciting new genomic island encoding toxins with putative activity on the actin cytoskeleton or the small GTPases.

The third cluster chosen for further phenotypic analysis (FIG. 2) carries a gene predicting a protein with putative phospholipase D activity (BPSS1381), neighbouring an endonuclease/exonuclease/phosphatase family protein (BPSS1382). This positive gene cluster also contains a putative membrane magnesium transporter protein. Macrophage monolayers treated with a preparation of clones (E. coli cells, whole cell lysate and supernatant) containing this region of interest show a dramatic decline in cell density, indicating a potent degree of cytotoxicity. Macrophages surviving such treatment become distended with little remaining cytoskeletal or nuclear structure.

The fourth cluster chosen for follow up analysis contains two CDSs encoding a putative hemagglutinin and with homology to the large filamentous hemagglutinin precursor, FhaB, (BPS S1727) and hemolysin activator-like protein precursor, FhaC (BPSS1728) of Bordetella pertussis (FIG. 2). The B. pertussis filamentous hemagglutinin (FHA) is an adhesin and facilitates attachment to the host cell during infection following secretion which is dependent on FhaC (Jacob-Dubuisson et al. (1996) Mol. Microbiol. 19:65-78). Alongside this role in attachment, FHA is also described as having accessory functions, including pro-apoptotic activity towards macrophages (Abramson et al. (2001) Infect. Immun 69:2650-2658). Macrophages exposed to preparations of clones containing the B. pseudomallei hemagglutinin-like gene display a very interesting phenotype with formation of dramatic actin projections or ‘tails’ extending towards neighbouring cells from shrunken cell bodies containing condensed nuclei.

Fractionation of Bioactivity from the Sy1A-Like Gene Cluster

Finally, to demonstrate how bioactivities from positive gene clusters can be further confirmed and fractionated, the inventors carried out a more detailed analysis of the gene cluster encoding the Burkholderia ‘Sy1A’ homolog, here termed By1A. The Sy1A gene product is a small molecule secreted into the supernatant of cultures of P. syringae bacteria (Waspi et al. (1998) The American Phytopathological Society 11:727-733). Cell-free supernatants from recombinant E. coli clones carrying the Burkholderia By1A encoding cluster also show strong cytotoxic activity and remaining macrophages from treated monolayers display a shrunken and rounded phenotype. Such activity is absent from the cytosolic fraction of the same preparations showing that the bioactive component, By1A, is secreted.

### Discussion

The bacterium B. pseudomallei has a complex and poorly understood infection cycle involving periods in the environment and periodic infection of man. Although a serious human pathogen, many of the virulence mechanisms of B. pseudomallei remain to be elucidated. Burkholderia infect and replicate within host macrophages and subsequently induce macrophage cell death, but the mechanisms whereby they affect anti-macrophage activity are not completely understood. The present application describes a gain of function screen which successfully and rapidly detected over 100 anti-macrophage encoding gene clusters within genomic libraries of B. pseudomallei expressed in recombinant E. coli. This screen pulls out genomic factors either equipping E. coli with toxic elements which kill macrophages or an improved ability to evade them or improve growth, thus killing the cells by overwhelming/nutrient depletion (e.g. hydrogen peroxide scavenging, drug export or biofilm formation).

### Cluster Composition and Likely Effectors

Phenotypic analysis of four regions of interest detected by the screen begin to link activity of the gene products to some of the important phenotypes associated with Burkholderia infection and may allow workers to begin to answer how bacterial cells persist within and spread between infected macrophages. Several of these positive gene clusters are therefore worthy of further discussion. The first such region contains homology to the B. pertussis filamentous hemagglutinin or FHA. FHA is an adhesin which facilitates attachment to host cells during infection. Alongside this role in attachment, FHA also has other accessory functions, including pro-apoptotic activity towards host macrophages (Abramson et al. (2001) Infect. Immun. 69:2650-2658) and it is suggested that this may be used to combat the host cell-mediated immune response whilst infection is being established. Positive clones recovered in the presently described screen, containing a B. pseudomallei region homologous to FHA, cause dramatic actin protrusions from macrophages and apoptotic nuclei. It should be noted that B. pseudomallei travel down actin protrusions in order to spread to neighbouring cells via actin based motility (Kespichayawattana et al. (2000) infect. Immun 68:5377-5384) and that the effector, BimA, has been observed as required for this activity as mutants in this gene do not induce formation of actin tails (Stevens et al. (2005) Mol. Microbiol. 56:40-53). These FHA homolog induced actin protrusions may therefore play a central role in regulating the actin cytoskeleton for adherence of B. pseudomallei to the host cell or perhaps act as an effector, like BimA, involved in the process of intracellular spread itself.

Following attachment, B. pseudomallei is capable of invading and replicating within both phagocytic and epithelial cells either entering via cell mediated phagocytosis or via the combined effects of Bsa T3SS and its internally delivered effector, BopE (Stevens et al. (2004) Microbiology 150:2669-2676). Once inside the host cell B. pseudomallei cells exhibit actin based motility and induce host cell fusion, resulting in the formation of ‘multi-nucleated giant cells’ or MNGCs. This disclosure describes both a MNGC-like and pro-apoptotic phenotype linked to a novel toxin cluster containing genes predicting proteins with homology to the Salmonella enterica virulence associated protein SpvB and different components of the Toxin complexes (Tcs) of the insect pathogen Photorhabdus luminescens. Whilst the inventors have not designated specific phenotypes to specific genes within this novel cluster, it is suggested that these genes are responsible for reorganization of the actin cytoskeleton, possibly directly or via effects on the small Rho GTPases. Further, this suggests that the formation of MNGCs in Burkholderia infected hosts can be induced by a single factor, independently of the actin-based motility mechanism. Further work addressing the process of secretion and mechanism of action of this novel toxin cluster will be important in understanding the role this region plays in MNGC formation.

Anti-macrophage activity is also seen in response to positive clones containing a phospholipase D domain protein. A PLD gene encoding a protein with phospholipase D activity is associated with phagosomal escape in Rickettsia prowazekii (Driskell et al. (2009) Infect. Immun. 77:3244-3248). Mutants of PLD in Rickettsia show attenuated virulence in guinea pigs and animals immunized with the mutant strain are protected from subsequent challenge with the wild-type strain. Phospholipase D is also a major virulence determinant of Corynebacterium pseudotuberculosis and plays a key role in macrophage death (McKean et al. (2007) Microbiology 153:2203-2211). Mutation of PLD genes in the pathogens Corynebacterium pseudotuberculosis and Rickettsia prowazekii have, therefore, shown promise as a strategy for development of attenuated strain vaccines (Driskell et al. (2009) Infect. Immun 77:3244-3248; Hodgson et al. (1999) Vaccine 17:802-808). Again, the role of the PLD-like gene in the interaction of Burkholderia with host macrophages therefore warrants further attention.

Finally, many of the positive gene clusters are associated with the production of NRPS/PKS systems which are predicted to make small molecules or peptides. Whilst it is often possible to predict the likely structure of the small molecules made via the unique combinations of PKS modules present, the role of these gene products in bacterial virulence is often less clear. The inventors focussed on one such positive region in B. pseudomallei which appears to encode a molecule similar to the proteome inhibitor Sy1A from P. syringae, which is termed By1A here. Sy1A is of extreme interest as it shows good activity against carcinoma derived cell lines and By1A may therefore be similarly interesting and important. The inventors have shown that gene clusters encoding By1A produce an active compound cytotoxic towards macrophages when expressed in recombinant E. coli. Moreover, this bioactivity can be localized to the supernatant of the recombinant E. coli culture suggesting that it does indeed correspond to a secreted small molecule similar to Sy1A.

TABLE 1
Complete inventory of anti-macrophage associated loci identified on B. pseudomallei K96243 chromosome 1

| | Genetic Region | | No. of | |
|---|---|---|---|---|
| Hit # | (bp) | CDS coordinates | clones | Features within region |
| 1 | 28900, 39500 | BPSL0027-BPSL0039 | 2 | Flagellar biosynthetic protein |
| | (10.6 kb) | | | Hypothetical protein |
| | | | | Methyltransferase |
| | | | | Two component regulatory system sensor kinase |
| | | | | protein |
| | | | | ABC transporter, ATP binding domain |
| | | | | ABC transporter # 84 = Class III importer OTCN |
| 2 | 49000, 62300 | BPSL0045-BPSL0056 | 3 | type III restriction system endonuclease (17f02 and |
| | (13.3 kb) | | | 16f04 only) |
| | | | | AraC family regulatory protein |
| | | | | Putative ABC transporter |
| | | | | putative phenylacetaldehyde dehydrogenase |
| | | | | conserved hypothetical protein |
| | | | | putative short chain dehydrogenase |
| | | | | glucose-methanol-choline (GMC) oxidoreductase |
| | | | | family protein |
| | | | | ABC transporter #29 = Class III importer HAA |
| 3 | 148600, 156200 | BPSL0130-BPSL0146 | 5 | ON GI 2 |
| | (7.6 kb) | | | Repeat region |
| | | | | Hypothetical protein BPSL0130 |
| | | | | zinc finger CHC2-family protein |
| | | | | Bacteriophage related proteins |
| 4 | 163300, 166000 | BPSL0152-BPSL0154 | 6 | ON GI 2 |
| | (2.7 kb) | | | Repeat region |
| | | | | Bacteriophage tail protein |
| 5 | 177400, 182900 | BPSL0173-BPSL0177 | 4 | ON GI 2 |
| | (5.5 kb) | | | Repeat region |
| | | | | Putative phage portal vertex protein |
| 6 | 298800, 315300 | BPSL0286-BPSL0298, | 2 | metal ion transporter, metal ion (Mn2+/Fe2+) |
| | (16.5 kb) | (partial) | | carbohydrate porin |
| | | | | natural resistance-associated macrophage protein |
| 7 | 332200, 358800 | BPSL0313-BPSL0335 | 2 | UDP-N-acetylglucosamine pyrophosphorylase |
| | (26.6 kb) | | | outer membrane porin |
| 8 | 475200-482200 | BPSL0436-BPSL0465 | 7 | phosphoenolpyruvate-protein phosphotransferase |
| | (7.0 kb) | | | glutamate--cysteine ligase |
| | 490900, 511000 | BPSL0452-BPSL0465 | 5 | cytochrome c oxidase polypeptide I |
| | (20.1 kb) | | | hypothetical protein BPSL0471 |
| | | | | error-prone DNA polymerase |
| | | | | ABC-type transporter, periplasmic component |
| | | | | ABC transporter BPSL0466 = Class III No 8 DLM |
| | | | | BP BPSL0467-69 = NEW1 unknown function |
| 9 | 616000, 624300 | BPSL0563-BPSL0568 | 3 | ON GI 3 |
| | (8.3 kb) | | | hypothetical protein BPSL0565 |
| | | | | von Willebrand factor type A |
| | | | | Subtilisin-like serine protease |
| | | | | phage integrase family protein |
| 10 | 648800, 664200 | BPSL0584-BPSL0591 | 2 | GI 3 OVERLAP |
| | (15.4 kb) | | | hypothetical protein BPSL0590, BPSL0591 |
| | | | | FG-GAP repeat/YD repeat/RHS repeat protein |
| | | | | phage integrase family protein |
| 11 | 705300, 730300 | BPSL0623-BPSL0644 | 2 | oxidoreductase, FAD-binding |
| | (25 kb) | | | |
| 12 | 771400, 780600 | BPSL0677-BPSL0683 | 5 | putative asparagine synthetase B |
| | (9.2 kb) | | | 3-phosphoshikimate 1-carboxyvinyltransferase |
| | | | | HAD-superfamily hydrolase |
| 13 | 817800, 832600 | BPSL0716-BPSL0723 | 3 | hypothetical protein BPSL0719 |
| | (14.8 kb) | | | glycosyl transferase family 51 |
| | | | | membrane carboxypeptidase |
| 14 | 868100, 894600 | BPSL0752-BPSL0769 | 2 | ON GI 4 |
| | (26.5 kb) | | | hypothetical protein BPSL0764 |
| | | | | SNF2-related: helicase |
| | | | | ATP phosphoribosyltransferase |
| | | | | putative phospholipase protein |
| 15 | 910300, 920600 | BPSL0783-BPSL0792 | 2 | peptidase |
| | (10.3 kb) | | | O-antigen polymerase family protein BPSL0791 |
| | | | | serine protease |
| 16 | 952000, 962100 | BPSL0818-BPSL0828 | 4 | glutathione dependent alcohol dehydrogenase |
| | (10.1 kb) | | | AraC family transcription regulator |
| | | | | PfkB family kinase |
| 17 | 981700, 991800 | BPSL0843-BPSL0846 | 2 | 2-hydropantoate 2-reductase |
| | (3.95 kb) | | | major facilitator family transporter |
| 18 | 1013300, 1047700 | BPSL0873-BPSL0897 | 2 | Drug resistance transporter EmrB/QacA |
| | (34.4 kb) | | | translation elongation factor G-sodB |
| 19 | 1105000, 1112400 | BPSL0945-BPSL0953 | 5 | ON GI 5 |
| | (7.4 kb) | | | putative type I restriction-modification methylase |
| | | | | hypothetical protein |
| | | | | putative replication protein |
| 20 | 1115300, 1132400 | BPSL0956-BPSL0973 | 2 | dihydroxy acid dehydratase |
| | (17.1 kb) | | | leucyl aminopeptidase |
| 21 | 1199900, 1213800 | BPSL1030-BPSL1043 | 2 | ABC transporter, ATP-binding protein |
| | (13.9 kb) | | | ABC transporter, permease protein |
| | | | | putative two-component system, sensor kinase |
| | | | | ABC transporters: |
| | | | | Class III Familiy OTCN #90 (BPSL1039- |
| | | | | BPSL1040) |
| | | | | Class III Family PAO #97 (BPSL1030-BPSL1033) |
| | 1218200, 123570 | BPSL1048-BPSL1068 | 2 | ABC transporter, ATP-binding protein |
| | (17.9 kb) | | | hypothetical proteins |
| | | | | ABC transporter Class II Family ART #2 |
| 22 | 1268200, 1289000 | BPSL1097-BPSL1110 | 2 | DNA translocase FtsK |
| | (20.8 kb) | | | cell division protein FtsK |
| | | | | glycosyl hydrolase, family 15 |
| | | | | intracellular PHB depolymerase |
| 23 | 1426000, 1439200 | BPSL1242-BPSL1247 | 2 | metallopeptidase, M24 family BPSL1242 |
| | (13.2 kb) | | | |
| | 1438600, 1450100 | BPSL1247-BPSL1254 | 3 | two-component hybrid sensor and regulator |
| | (11.5 kb) | | | D-3-phosphoglycerate dehydrogenase |
| | | | | metallopeptidase family M24 BPSL1247 |
| | 1450100, 1469000 | BPSL1255-BPSL1267 | 2 | CdpA BPSL1263 |
| | (18.9 kb) | | | putative transport system/multidrug resistance |
| | | | | protein MdtB/AcrB, AcrD, AcrF family protein |
| | | | | (BPSL1266) |
| | | | | putative transport system membrane protein |
| | | | | (BPSL127) |
| 24 | 1515600, 1528600 | BPSL1301-BPSL1309 | 2 | GGDEF domain-containing protein |
| | (13 kb) | | | 200 kDa antigen p200, putative |
| | | | | ABC transporter, periplasmic substrate-binding |
| | | | | protein |
| | | | | diguanylate cyclase |
| | | | | LacI family transcriptional regulator |
| | | | | extracellular solute-binding protein |
| | | | | non-ribosomal peptide synthetase modules and |
| | | | | related proteins-like, putative |
| | | | | hypothetical proteins |
| | | | | ABC transporter BPSL1301-BPSL1303 Class III |
| | | | | transporter, #45 Family: MOI |
| 25 | 1580600, 1597500 | BPSL1353-BPSL1367 | 2 | polyphosphate kinase |
| | (16.9 kb) | | | FtsH endopeptidase/ATP-dependent metalloprotease |
| | | | | FtsH BPSL1356 |
| | | | | phosphoglucosamine mutase |
| 26 | 1614900, 1618300 | BPSL1385-BPSL1390 | 3 | ON GI 7 |
| | (3.4 kb) | | | Hypothetical proteins |
| | 1618900, 1626000 | BPSL1392-BPSL1398 | 2 | putative exported endonuclease BPSL1394 |
| | (7.1 kb) | | | outer membrane porin BPSL1397 |
| | | | | histone deacetylase family, putative BPSL1395 |
| 27 | 1689000, 1701700 | BPSL1452-BPSL1465 | 3 | replicative DNA helicase |
| | (12.7 kb) | | | hypothetical protein BURPS1710b_2420 |
| | | | | Ser/Thr protein phosphatase family protein |
| | | | | family C40 unassigned family peptidase BPSL1465 |
| 28 | 1702600, 1716300 | BPSL1467-BPSL1478 | 2 | dolichyl-phosphate-mannose-protein |
| | (13.7 kb) | | | mannosyltransferase family protein |
| | | | | (BPSL1474) (glycosyltransferase) |
| | | | | threonine synthase |
| | | | | aminotransferase AlaT |
| | | | | homoserine dehydrogenase |
| | | | | putative LPS biosynthesis-related protein |
| 29 | 1717300, 1730900 | BPSL1479-BPSL1492 | 2 | ClpB ATPase dependent protease, chaperonin |
| | (13.6 kb) | | | |
| 30 | 1791900, 1800900 | BPSL1538-BPSL1551 | 3 | MiaB-like tRNA modifying enzyme YliG |
| | (18.5 kb) | | | ABC transporter, ATP-binding protein |
| | | | | AsmA family protein |
| | | | | aldehyde dehydrogenase (NAD) family protein |
| | | | | hypothetical proteins |
| | | | | ABC transporter BPSL1545-1546 Class III ABC |
| | | | | transporter Family NO (unclassified) #68 |
| | | | | BPSL1548 - Class II ABC transporter Family |
| | | | | ART REG |
| 31 | 1811400, 1822100 | BPSL1561-BPSL1567 | 3 | AcrB/AcrD/AcrF family protein |
| | (10.7 kb) | | | RND family multidrug efflux pump |
| | | | | acriflavin reistance protein |
| | | | | putative voltage-gated ClC-type chloride channel |
| | | | | ClcB BPSL1570 (1h11 and 15f11 only, hit extends to |
| | | | | BPSL1575) |
| 32 | 1895300, 1906400 | BPSL1634-BPSL1642 | 3 | OVERLAPS GI 8 |
| | (9.601 kb) | | | putative two-component regulatory system |
| | | | | (BPSL1634) |
| | | | | GGDEF -like protein BPSL1635 |
| | | | | lipase (BPSL1637) |
| | | | | putative transposase |
| | | | | GntR family regulatory protein (BPSL1642) |
| 33 | 1906600, 1922900 | BPSL1644-BPSL1659 | 3 | ON GI 8 |
| | (16.3 kb) | | | succinate-semialdehyde dehydrogenase |
| | | | | outer membrane porin |
| | | | | ABC transporter, ATP binding protein |
| | | | | ABC transporter BPSL1649-BPSL1652 = Class III |
| | | | | ABC transporter Family MOI #50 |
| 34 | 1943800, 1960800 | BPSL1667-BPSL1679 | 2 | ON GI 8 |
| | (16.8 kb) | | | hypothetical proteins |
| | | | | tetratricopeptide repeat protein |
| | | | | major facilitator family transporter |
| | | | | putative two component system , response regulator |
| | | | | (BPSL1669) |
| | | | | outer membrane porin (BPSL1674) |
| 35 | 1983700-1989700 | BPSL1705-BPSL1706 | 2 | ON GI 8 |
| | (6 kb) | | | tash protein pest motif family |
| | | | | hemagglutinin |
| | | | | Hep_Hag family |
| | | | | YadA domain-containing protein |
| | | | | Autotransporter adhesion |
| | | | | putative HNS-like protein (BPSL1706) |
| 36 | 1988935-2019700 | BPSL1707-BPSL1714 | 3 | putative syringomycin synthetase/nonribosomal |
| | (30.8 kb) | | | peptide synthetase (BPSL1712) |
| | | | | putative penicillin amidase (BPSL1710) |
| | | | | carbamoyltransferase family protein (BPSL1711) |
| | | | | family S45 unassigned peptidase |
| | | | | BPSL1710-1714 = beginning of NRPS cluster- |
| | | | | entire cluster not covered |
| 37 | 2230900-2237500 | BPSL1875-BPSL1878 | 2 | methyl-accepting chemotaxis protein (BPSL1875) |
| | (6.6 kb) | | | putative phospholipase (BPSL1876) |
| | | | | phosphoesterase family protein |
| 38 | 2634300, 2647900 | BPSL2195-BPSL2204 | 2 | ABC transporter Class III #75 family OPN |
| | (13.6 kb) | | | |
| 39 | 2672100, 2691000 | BPSL2228-BPSL2234 | 2 | amino acid adenylation domain protein |
| | (18.8 kb) | | | non-ribosomal peptide synthetase/syringomycin |
| | | | | synthetase (BPSL2229 & BPSL2232) |
| | | | | major facilitator family transporter |
| | | | | linear gramicidin synthetase subunit D |
| | | | | multidrug efflux RND membrane fusion protein |
| | | | | BPSL2214-BPSL2233 = putative NRPS cluster. |
| | | | | Linked to putative efflux transport genes |
| | 2681600, 2704400 | BPSL2230-BPSL2241 | 2 | AcrB/AcrD/AcrF family protein (BSPL2235) |
| | (22.8 kb) | | | putative exported lipase (BPSL2237) |
| | | | | outer membrane autotransporter |
| | | | | putative non-ribosomal peptide synthase |
| | | | | (BPSL2230-2241) |
| | | | | amino acid adenylation domain protein |
| | | | | acriflavin resistance protein |
| | | | | RND efflux transporter |
| | | | | multidrug ABC transporter (not on system |
| | | | | inventory) |
| 40 | 2719400, 2723900 | BSPL2255-BPSL2257 | 3 | cupin superfamily protein family |
| | (4.5 kb) | | | putative lipoprotein (BPSL2257) |
| | | | | metallo-beta-lactamase family protein |
| 41 | 2774600, 2788300 | BPSL2302-BPSL2309 | 4 | Response regulator protein (BPSL2303) |
| | (13.7 kb) | | | oligopeptidase A (BPSL2305) |
| | | | | putative nitrite extrusion proteins (BPSL2307-2308) |
| | | | | nitrate reductase, alpha subunit (BPSL2309) |
| | 2787600-2795400 | BPSL2310-BPSL2314 | 2 | Putative nitrate reductase; beta, delta and gamma |
| | (7.8 kb) | | | subunits (BPSL2310, 2311, 2312) |
| | | | | putative nitrate/nitrite sensor protein (BPSL2313) |
| | | | | Putative response regulator protein (BPSL2314) |
| | 2795400, 2821100 | BPSL2315-BPSL2334 | 3 | endonuclease, exonuclease, phosphatase family |
| | (26.5 kb) | | | BPSL2315 |
| | | | | putative nitrogen regulation protein NR (I) |
| | | | | BPSL2316 |
| | | | | putative nitrogen regulation protein NR (II) |
| | | | | BSPL2317 |
| | | | | glutamine synthetase ‘glnA’ BPSL2318 |
| | | | | putative membrane protein BPSL2321, 2322 |
| | | | | putative exported protein BPSL2323 |
| | | | | putative ATP-dependent helicase BPSL2324 |
| | | | | putative amino-acid acetyltransferase BPSL2325 |
| | | | | putative transporter protein BPSL2327 |
| | | | | CAIB/BAIF family protein BPSL2328 |
| | | | | putative acyl-CoA dehydrogenase BPSL2329 |
| | | | | LysR family regulatory protein BPSL2330 |
| | | | | hypothetical protein BPSL2319, BPSL2320, |
| | | | | BPSL2326, BPSL2331, BPSL2332, BPSL2333, |
| | | | | BPSL2334 |
| | 2818700, 2821200 | BPSL2332-BPSL2334 | 3 | hypothetical proteins |
| | (1.97 kb) | | | StaB |
| | | | | plasmid stabilisation system |
| | | | | addiction module toxin, RelE/StbE family |
| | | | | addiction module antitoxin |
| 42 | 2838300, 2850500 | BPSL2349-BPSL2356 | 4 | deoxyribodipyrimidine photolyase |
| | (12.2 kb) | | | alkane 1-monooxygenase |
| | | | | putative nitric oxide reductase |
| 43 | 2851300, 2862300 | BPSL2358-BPSL2367 | 4 | U32 family peptidase (BPSL2362) |
| | (11 kb) | | | coproporphyrinogen III oxidase (BPSL2366) |
| | | | | methyl-accepting chemotaxis protein (BPSL2367) |
| | | | | hypothetical proteins |
| 44 | 3059900, 3069300 | BPSL2538-BPSL2544 | 2 | adenosine deaminase |
| | (9.4 kb) | | | aminopeptidase N |
| | | | | hypothetical proteins |
| | | | | xanthine/uracil permease family protein |
| 45 | 3082100, 3089100 | BPSL2553-BPSL2559 | 3 | outer membrane porin protein |
| | (7.0 kb) | | | exonuclease, DNA polymerase III, epsilon |
| | | | | subunit family/GIY-YIG catalytic domain protein |
| | | | | putative siderophore receptor protein |
| | | | | TonB-dependent receptor |
| 46 | 3088900, 3096300 | BPSL2560-BPSL2566 | 2 | GTP pyrophosphokinase |
| | (7.4 kb) | | | (p)ppGpp synthetase I, SpoT/RelA |
| 47 | 3141200, 3157600 | BPSL2615-BPSL2630 | 2 | ABC transporter Class III family PAO #93 |
| | (18.2 kb) | | | (BPSL2615-2617) |
| | | | | UbiD family decarboxylase |
| 48 | 3172500, 3175700 | BPSL2650-BPSL2652 | 3 | ABC transporter Class III family HAA # 28 |
| | (3.2 kb) | | | (BPSL2561 and 2562 partial) |
| | 3175900, 3186000 | BPSL2652-BPSL2662 | 2 | urease subunits and accessory proteins |
| | (10.1 kb) | | | |
| 49 | 3200300, 3206900 | BPSL2675-BPSL2679 | 3 | wbiF—putative glycosyl transferase |
| | (6.6 kb) | | | wbiE—putative glycosyl transferase |
| | | | | wbiD—putative O-antigen methyl transferase |
| | | | | wbiC—putative glycosyl transferase |
| | 3207500-3232800 | BPSL2680-BPSL2702 | 2 | wzt ABC transporter ATP binding component |
| | (25.3 kb) | | | wzm ABC transporter membrane permease |
| | | | | rm1D |
| | | | | rm1C |
| | | | | rm1A |
| | | | | dTDP-glucose 4.6-dehydratase rm1B |
| | | | | putative 1-acyl-SN-glycerol-3-phosphate |
| | | | | acytransferase plsC |
| | | | | dihydroorotase-like protein pyrX |
| | | | | pyrB aspartate carbamoyltransferase BPS2690 |
| | | | | pyrR bifunctional regulator/uracil |
| | | | | phosphoribosyltransferase BPSL2691 |
| | | | | Chaperonins groEL and groES1 |
| | | | | Putative kinase BPSL2696 |
| | | | | ABC transporter Class I ABC transporter Family |
| | | | | CLS #7 |
| 50 | 3372100, 3384000 | BPSL2821-BPSL2830 | 2 | molecular chaperone DnaK |
| | (11.9 kb) | | | para-aminobenzoate synthase, component I PabB |
| | | | | BPSL2825 |
| 51 | 3473600, 3489600 | BPSL2904-BPSL2921 | 2 | tyrosyl-tRNA synthetase |
| | (16 kb) | | | anhydro-N-acetylmuramic acid kinase |
| 52 | 3589600, 3593500 | BPSL3012-BPSL3015 | 3 | glutamate N-acetyltransferase/amino-acid |
| | (3.0 kb) | | | acetyltransferase argJ |
| 53 | 3685000, 3696700 | BPSL3078-BPSL3096 | 2 | CspD cold shock-like protein BPSL3079 |
| | (26.9 kb) | | | Putative DnaK-type chaperone BPSL3080 |
| | | | | peptidase, M1 family BPSL3089 |
| | | | | colicin V processing peptidase BPSL3093 |
| | | | | putative toxin secretion ABC transporter |
| | | | | putative bacteriophage related peptidase BPSL3096 |
| | | | | TolC family type I secretion outer membrane |
| | | | | protein |
| | | | | ABC transporter Class I Family DPL # 11 |
| | | | | (BPSL3092-3094) |
| 54 | 3708600, 3714100 | BPSL3101-BPSL3117 | 2 | ON GI 10 |
| | (16.7 kb) | | | ClpB protease associated ATPase (partial) |
| | | | | BPSL3101 |
| | | | | Hypothetical proteins/membrane proteins |
| | | | | type VI secretion protein (EvpB family?) |
| | | | | Repeat regions BPSL3114-3117 |
| | | | | BPSL3103 and 3110 Type VI secretion (tss-1 = |
| | | | | BPSL3111-3097) |
| 55 | 3727100, 3732800 | BPSL3122-BPSL3127 | 4 | Cytochrome b/b6, N-terminal domain petB |
| | (5.7 kb) | | | BPSL3122 |
| | | | | serine protease DegQ (BPSL3125) |
| | | | | Sec-independent protein translocase proteins tatC |
| | | | | and tatB |
| 56 | 3782400-3802000 | BPSL3177-BPSL3198 | 2 | engB putative GTP-binding cell division protein |
| | (19.6 kb) | | | (BPSL3182) |
| | | | | preprotein translocase, SecY subunit BPSL3193 |
| | | | | DNA-directed RNA polymerase subunit alpha |
| 57 | 3813600, 3827500 | BPSL3219-BPSL3225 | 3 | DNA-directed RNA polymerase subunit beta |
| | (13.9 kb) | | | |
| 58 | 3881700, 3895500 | BPSL3265-BPSL3279 | 2 | Overlaps GI 11 |
| | (13.8 kb) | | | putative plasmid replication protein (BPSL3270) |
| | | | | hypothetical proteins |
| | | | | rare lipoprotein A family protein (rlpA BPSL3276) |
| | | | | metallo-beta-lactamase family protein (BPSL3277) |
| | | | | putative phospholipid-binding protein |
| | | | | cation efflux family protein |
| 59 | 3978600, 4012700 | BPSL3356-BPSL3380 | 3 | ATP-dependent DNA helicase Rep |
| | (34.1 kb) | | | glycine cleavage system proteins gcvT, gcvH, gcvP |
| | | | | putative heavy metal resistance membrane ATPase |
| | | | | BPSL3378 |

TABLE 2
Complete inventory of anti-macrophage associated loci identified on B. pseudomallei K96243 chromosome 2

| | Genetic region | | No. of | |
|---|---|---|---|---|
| Hit # | (bp) | CDS coordinates | clones | Features within region |
| 1 | 1, 8700 | BPSS0001-BPSS0009 | 3 | 2-amino-3-ketobutyrate coenzyme A ligase (Kbl) |
| | (8.7 kb) | | | BPSS0005 |
| | | | | phage integrase family protein |
| 2 | 49400, 60600 | BPSL0051-BPSL0058 | 2 | chromate resistance transport protein Chr A |
| | (11.2 kb) | | | BPSS0053 |
| | | | | chromate resistance exported protein ChrB |
| | | | | BPSS0054 |
| | | | | UvrA excinuclease ABC, A subunit BPSS0058 |
| | | | | (partial) |
| | | | | ABC transporter |
| | | | | Class III Family UVR #105 |
| 3 | 75300, 81200 | BPSS0066-BPSS0070 | 3 | non-hemolytic phospholipase C precursor |
| | (5.9 kb) | | | (BPSS0067) |
| | | | | twin-arginine translocation pathway (BPSS0066) |
| | | | | integrase catalytic region |
| 4 | 87100, 112400 | BPSS0077A-BPSS0092 | 5 | hypothetical protein BPSS0078 |
| | (25.3 kb) | | | Rhs element Vgr protein |
| | | | | cytochrome c oxidase, subunit I |
| | | | | Type V secretory pathway, adhesin AidA |
| | | | | PqaA protein |
| | | | | Hep_Hag family protein BPSS0088 |
| | | | | hypothetical proteins |
| | | | | Type I fimbriae BPSS0091 and BPSS0092 (partial) |
| 5 | 116000, 122000 | BPSS0096-BPSS0101 | 3 | hypothetical protein BPSS0098 |
| | (6.0 kb) | | | EvpB family type VI secretion protein |
| | | | | OmpA family protein |
| | | | | putative cytoplasmic protein |
| | | | | BPSS0095-BPSS0116 = Type VI secretion system-2 |
| 6 | 127800, 133200 | BPSS0104-BPSS0105 | 3 | hypothetical protein BPSS0104 |
| | (5.4 kb) | | | Rhs element Vgr protein BPSS0105 |
| 7 | 182500, 191700 | BPSS0142-BPSS0145 | 2 | putative glucan 1,4-alpha-glucosidase |
| | (9.2 kb) | | | putative amylase |
| | | | | ribose ABC transporter, ATP-binding protein |
| | | | | ATP-dependent RNA helicase DbpA |
| | | | | DEAD/DEAH box helicase domain protein |
| | | | | ABCtransporter Class III Family MOS # 58 |
| | | | | BPSL0140-0142 |
| 8 | 217300, 239600 | BPSS0165-BPSS0178 | 2 | type VI ImcF/SciS family protein BPSS0167 |
| | (22.3 kb) | | | OmpA family membrane protein BPSS0168 |
| | | | | chaperone clpB BPSS0174 |
| | | | | Putative lipoprotein BPSS0170 |
| | | | | EvpB family type VI secretion |
| | | | | Type VI secretion system-3 = BPSS0185-BPSS0167 |
| | 239700, 243400 | BPSS0179-BPSS0181 | 3 | SciB protein BPSS0179 |
| | (3.7 kb) | | | ImpA family type VI associated protein BPSS0180 |
| | | | | Rhs element Vgr protein BPSS0181 |
| 9 | 284300, 299200 | BPSS0210-BPSS0221A | 3 | radical SAM domain protein |
| | (14.9 kb) | | | drug resistance transporter, EmrB/QacA family |
| | | | | methyl-accepting chemotaxis protein tar BPSS0215 |
| | | | | N-acylglucosamine 2-epimerase |
| 10 | 310400, 316900 | BPSS0228-BPSS0232 | 4 | hypothetical protein BPSS0229 |
| | (6.5 kb) | | | type I phosphodiesterase |
| | | | | Glyoxalase/Bleomycin resistance protein/Dioxygenase |
| | | | | superfamily protein BPSS0230 |
| | | | | squalene/phytoene synthase family protein BPSS0232 |
| | 316300, 333200 | BPSS0233-BPSS0244 | 2 | TonB-dependent hemoglobin/transferrin/lactoferrin |
| | (16.9 kb) | | | receptor family protein |
| | | | | penicillin-binding protein, 1A family BPSS0238 |
| | | | | putative hemin ABC transport system, ATP-binding |
| | | | | protein BPSS0240 |
| | | | | putative hemin ABC transport system, membrane |
| | | | | protein BPSS0241 |
| | | | | putative hemin transport system, |
| | | | | substrate-binding protein BPSS0242 |
| | | | | putative hemin ABC transport system-related protein |
| | | | | BPSS0243 |
| | | | | putative exported heme receptor protein BPSS0244 |
| | | | | ABC transporter Class III Family ISVH #38 |
| 11 | 447200, 472700 | BPSS0320-BPSS0339 | 2 | amino acid dioxygenase BPSS0339 |
| | (25.5 kb) | | | iron-sulfur binding protein BPSS0322 |
| | | | | amino acid transporter, putative BPSS0330 |
| | | | | aldehyde dehydrogenase (NAD) family protein |
| | | | | BPSS0329 |
| 12 | 532400, 543600 | BPSS0386-BPSS0402 | 3 | Overlaps GI 13 |
| | (11.2 kb) | | | hypothetical proteins |
| | | | | bacteriophage/transposase fusion protein |
| 13 | 562800, 570400 | BPSS0410-BPSS0414 | 2 | Hypothetical proteins BPSS0410 to 413 |
| | (7.6 kb) | | | acetolactate synthase |
| 14 | 617100, 633800 | BPSS0452-BPSS0463 | 2 | helix-hairpin-helix motif domain/PHP domain protein |
| | (16.7 kb) | | | multicopper oxidase family protein BPSS0456 |
| | | | | FAD-dependent oxidoreductase |
| | | | | putative copper-resistance exported protein |
| | | | | BPSS0458 |
| | | | | putative copper-resistance membrane protein |
| | | | | BPSS0459 |
| | | | | putative methyl-accepting chemotaxis protein |
| | | | | BPSS0460 |
| | | | | hypothetical protein BPSS0463 |
| | 634500, 638100 | BPSS0464-BPSS0468 | 3 | putrescine ABC transporter, ATP-binding protein |
| | (3.6 kb) | | | putrescine ABC transporter, periplasmic putrescine- |
| | | | | binding protein |
| | | | | potH, potG, potF |
| | | | | ABC transporter Class III Family MOI #52 |
| 15 | 666700, 674400 | BPSS0491-BPSS0496 | 3 | alkyl hydroperoxide reductase subunit |
| | (7.7 kb) | | | chitin binding domain-containing protein |
| 16 | 717100, 719600 | BPSS0525-BPSS0526 | 2 | hypothetical protein BPSS0525 |
| | (2.5 kb) | | | |
| 17 | 731800, 750000 | BPSS0537-BPSS0548 | 3 | putative glycosyl transferase BPSS0537 |
| | (18.2 kb) | | | drug resistance transporter, EmrB/QacA family |
| | | | | protein (HylD) BPSS0541 |
| | | | | glycosyl hydrolase BPSS0542 |
| | | | | levanase |
| | | | | glutathione-independent formaldehyde |
| | | | | dehydrogenase |
| | | | | transcriptional regulator, AraC family |
| | | | | serine hydroxymethyltransferase |
| | 749900, 763200 | BPSS0549-BPSS0558 | 4 | NADH: flavin oxidoreductase/NADH oxidase family |
| | (13.3 kb) | | | protein |
| | | | | iron-sulfur cluster binding protein |
| | | | | hypothetical protein BURPS1710b_A2114 |
| | | | | amino acid permease |
| 18 | 811900, 833600 | BPSS0591-BPSS0610 | 3 | TonB-dependepnt Fe(III)-pyochelin receptor |
| | (21.7 kb) | | | iron-regulated membrane protein |
| | | | | siderophore transporter, RhtX/FptX family |
| | | | | sigma-54 activated regulatory protein |
| | | | | permease protein |
| | | | | LysR family regulatory protein |
| | | | | aldehyde dehydrogenase (NAD) family protein |
| | | | | TauD/TfdA family dioxygenase |
| | | | | thiamine pyrophosphate enzyme family protein |
| | | | | phosphoenolpyruvate phosphomutase |
| | | | | putative hydrolase |
| 19 | 853500, 885000 | BPSS0626-BPSS0651 | 2 | putative drug efflux protein BPSS0625 |
| | (31.5 kb) | | | phospholipase, patatin family BPSS0632 |
| | | | | amylo-alpha-1,6-glucosidase |
| | | | | peptidase |
| | | | | ‘mreB’ rod shape-determining protein BPSS0633 |
| | | | | glyoxalase/bleomycin resistance protein/dioxygenase |
| | | | | superfamily protein BPSS0639 |
| | | | | sensor kinase protein BPSS0642 |
| | | | | AraC family regulatory protein BPSS0648 |
| 20 | 925100, 938400 | BPSS0685-BPSS0696 | 3 | monoxygenase |
| | (13.3 kb) | | | sensor kinase protein BPSS0687 |
| | | | | 5-carboxymethyl-2-hydroxymuconate semialdehyde |
| | | | | dehydrogenase |
| 21 | 1060000, 1070400 | BPSS0794-BPSS0797 | 4 | oxidoreductase, short chain dehydrogenase/reductase |
| | (10.4 kb) | | | family |
| | | | | hypothetical protein BURPS668_A1165 |
| | | | | hemagglutinin family protein/Hep_Hag family |
| | | | | protein BPSS0796 |
| | | | | chromosome segregation ATPase |
| | | | | IclR family regulatory protein |
| 22 | 1075600, 1091400 | BPSS0803-BPSS0813 | 2 | Putative hemolysin BPSS0803 |
| | (15.8 kb) | | | diguanylate cyclase/cyclic diguanylate |
| | | | | phosphodiesterase |
| | | | | aromatic amino acid transport protein |
| | | | | aromatic amino acid aminotransferase |
| | | | | hypothetical protein BPSS0812 |
| | | | | sensor histidine kinase BPSS0813 |
| | 1091200, 1104100 | BPSS0814-BPSS0819 | 3 | putative chemotaxis protein BPSS0814 |
| | (12.9 kb) | | | penicillin-binding protein 1C BPSS0816 |
| | | | | alpha-2-macroglobulin domain protein BPSS0817 |
| | 1110400, 1128100 | BPSS0825-BPSS0840 | 3 | isopenicillin N epimerase BPSS0826 |
| | (17.7 kb) | | | microbial collagenase |
| | | | | monooxygenase family protein |
| | | | | universal stress family protein |
| | | | | oxidoreductase, zinc-binding dehydrogenase family |
| | | | | Collagenase = subfamiliy M9A unassigned peptidase |
| | | | | with similarity to Vibrio parahemolyticus PrtVp |
| 23 | 1258900, 1270000 | BPSS0957-BPSS0961 | 2 | hypothetical protein BPSS0957 |
| | (11.1 kb) | | | Rhs element Vgr protein BPSS0958 |
| | | | | Rhs-related membrane protein/YD repeat protein |
| | | | | BPSS0960 |
| | | | | BPSS0958 not previously documented as a Vgr-like |
| | | | | protein in K96243 |
| 24 | 1275000, 1295000 | BPSS0965-BPSS0976 | 3 | oxidoreductase alpha (molybdopterin) subunit |
| | (20 kb) | | | BPSS0969 |
| | | | | GntR family transcriptional regulator BPSS0970 |
| | | | | Mg(2+) transport ATPase, P-type 2 ‘mgtB’ |
| | | | | BPSS0973 |
| | | | | Subfamily S8A unassigned peptidase BPSS0974 |
| 25 | 1302600, 1315600 | BPSS0984-BPSS0995 | 2 | UDP-N-acetylglucosamine 1- |
| | (13 kb) | | | carboxyvinyltransferase, putative BPSS0984 |
| | | | | major facilitator family transporter |
| | | | | histidine kinase BPSS0990 |
| | | | | Response regulator BPSS0991 |
| | | | | catalase ‘katB’ BPSS0993 |
| | | | | methyltransferase family protein |
| | | | | AraC family transcriptional regulator BPSS0995 |
| 26 | 1363200, 1395300 | BPSS1008-BPSS1020 | 2 | polyketide synthase (PksK) BPSS1008 |
| | (32.1 kb) | | | polyketide synthase BPSS1009 |
| | | | | putative halogenase BPSS1010 |
| | | | | putative membrane transport protein BPSS1011 |
| | | | | putative voltage-gated clc-type chloride channel |
| | | | | BPSS1012 |
| | | | | putative LysR family transcriptional regulator |
| | | | | BPSS1015 |
| | | | | putative ionic antiporter BPSS1016 |
| | | | | putative antibiotic resistance protein BPSS1017 |
| | | | | putative fumarylpyruvate hydrolase BPSS1019 |
| | | | | putative AraC family transcriptional regulator |
| | | | | BPSS1020 |
| 27 | 1418000, 1437100 | BPSS1041-BPSS1059 | 3 | On GI 15 |
| | (19.1 kb) | | | Repeat region |
| | | | | heavy metal efflux pump CzcA BPSS1041 |
| | | | | bacteriophage replication gene A protein (GpA) |
| | 1418000, 1442100 | BPSS1059-BPSS1065 | 2+ | Repeat region |
| | (24.1 kb) | | | Bacteriophage related proteins |
| | | | | (many orphan end sequence alignments) |
| 28 | 1460300, 1482200 | BPSS1089-BPSS1102 | 2 | bacteriophage late control gene D protein BPSS1089 |
| | (21.9 kb) | | | alanine dehydrogenase BPSS1090 |
| | | | | putative capsule biosynthesis protein BPSS1097 |
| | | | | heat-shock chaperone protein BPSS1095-BPSS1096 |
| | | | | cation transport ATPase protein BPSS1100 |
| | | | | putative methyltransferase |
| 29 | 1488000, 1494800 | BPSS1110-BPSS1114 | 3 | putative gamma-butyrobetaine,2-oxoglutarate |
| | (6.8 kb) | | | dioxygenase BPSS1110 |
| | | | | hypothetical protein |
| | | | | transporter, basic amino acid/polyamine antiporter |
| | | | | BPSS1112 |
| | | | | Family S54 unassigned peptidase BPSS1113 |
| | | | | ATP-dependent metalloprotease FtsH BPSS1114 |
| 30 | 1505400, 1523600 | BPSS1121-BPSS1135 | 2 | putative GTP cyclohydrolase protein BPSS1121 |
| | (18.2 kb) | | | WD repeat-containing protein BPSS1122 |
| | | | | FAD/FMN-binding/pyridine nucleotide-disulphide |
| | | | | oxidoreductase family protein |
| | | | | putative riboflavin biosynthesis protein BPSS1125 |
| | | | | putative O-methyltransferase BPSS1126 |
| | | | | putative transmembrane transporter protein |
| | | | | BPSS1128 |
| | | | | putative LuxR family transcriptional regulator |
| | | | | BPSS1131 |
| | | | | putative 2,4-dienoyl-CoA reductaseBPSS1133 |
| | | | | PadR-like family regulatory protein BPSS1134 |
| | | | | putative hydroxyethylthioazole kinase BPSS1135 |
| 31 | 1695900, 1703600 | BPSS1252-BPSS1257 | 3 | hypothetical proteins |
| | (7.7 kb) | | | ribonuclease E |
| | | | | major facilitator transporter family protein BPSS1252 |
| | 1704000, 1722700 | BPSS1258-BPSS1267 | 4 | putative oxidoreductase/dehydrogenase BPSS1258 |
| | (18.7 kb) | | | alcohol dehydrogenase, iron-containing BPSS1259 |
| | | | | hypothetical protein |
| | | | | OmpA family membrane protein BPSS1264 |
| | | | | putative peptide/siderophore synthetase BPSS1266 |
| | | | | putative MbtH-like protein BPSS1267 |
| | | | | (Methyl-accepting chemotaxis protein) |
| | 1719700, 1722600 | BPSS1267-BPSS1269 | 5 | putative MbtH-like protein BPSS1267 |
| | (2.9 kb) | | | putative efflux system protein BPSS1268 |
| | | | | non-ribosomal peptide synthase BPSS1269 |
| | | | | (syringomycin synthetase) |
| | 1719600, 1727800 | BPSS1267-BPSS1269 | 2 | putative MbtH-like protein BPSS1267 |
| | (8.2 kb) | | | putative efflux system protein BPSS1268 |
| | | | | non-ribosomal peptide synthase BPSS1269 |
| | | | | (syringomycin synthetase) |
| 32 | 1858200, 1864800 | BPSS1358-BPSS1362 | 3 | putative sensor kinase protein BPSS1358 |
| | (6.6 kb) | | | putative response regulator protein BPSS1359 |
| | | | | putative two-component sensor kinase protein |
| | | | | BPSS1360 |
| | | | | glyoxylate reductase |
| 33 | 1883600, 1891900 | BPSS1377-BPSS1384 | 6 | cytochrome c oxidase BPSS1377 |
| | (8.3 kb) | | | putative magnesium transporter BPSS1379 |
| | | | | Phospholipase D domain protein BPSS1381 |
| | | | | endonuclease/exonuclease/phosphatase family protein |
| | | | | BPSS1382 |
| | | | | rotamase BPSS1383A |
| | | | | hypothetical protein |
| 34 | 1935000, 1940000 | BPSS1422-BPSS1425 | 2 | beta-hydroxylase |
| | (5.0 kb) | | | glycine betaine/L-proline ABC transporter, |
| | | | | periplasmic glycine betaine/L-proline-binding protein |
| | | | | BPSS1423, BPSS1425 |
| | | | | transcriptional regulator, AraC family protein |
| | | | | BPSS1424 |
| | | | | ABC transporter Class III family OTCN # 88 |
| | | | | BPSS1423+ 1425 |
| 35 | 1981200, 1994900 | BPSS1453-BPSS1462 | 4 | iron-sulfur cluster-binding protein, rieske |
| | (13.7 kb) | | | family/putative carboxynorspermidine decarboxylase |
| | | | | hypothetical protein |
| | | | | major facilitator family transporter BPSS1458 |
| | | | | two-component sensor kinase BPSS1460 |
| | | | | putative two-component response regulator |
| | | | | BPSS1461 |
| 36 | 2015300, 2035000 | BPSS1476-BPSS1492 | 4 | hypothetical protein |
| | (19.7 kb) | | | putative GntR family transcriptional regulator protein |
| | | | | BPSS1477 |
| | | | | Mandelate racemase/muconate lactonizing enzyme, |
| | | | | C-terminal domain protein BPSS1478 |
| | | | | putative dehydrogenase BPSS1478A |
| | | | | hypothetical protein |
| | | | | putative NH(3)-dependent NAD(+) synthetase |
| | | | | BPSS1482 |
| | | | | putative TetR family transcriptional regulator |
| | | | | BPSS1483 |
| | | | | copper-containing nitrite reductase BPSS1487 |
| | | | | N-acetylmuramoyl-L-alanine amidase domain- |
| | | | | containing protein BPSS1490 |
| | | | | glycosyltransferase TibC BPSS1491 |
| 37 | 2110900, 2127300 | BPSS1556-BPSS1566 | 2 | metabolite: proton symporter family protein |
| | (16.4 kb) | | | dehydrogenase, FMN-dependent family |
| | | | | MarR-family transcriptional regulator BPSS1556 |
| | | | | alpha-ketoglutarate permease ‘kgtP’ BPSS1558 |
| | | | | LysR-family transcriptional regulator BPSS1559 |
| | | | | serine carboxypeptidase family protein BPSS1561 |
| | | | | serine protease, kumamolysin BPSS1562 |
| | | | | phosphate transporter BPSS1566 |
| 38 | 2333600, 2359100 | BPSS1699-BPSS1719 | 4 | tryptophan synthase beta chain ‘trpB’ BPSS1699 |
| | (25.5 kb) | | | 2-oxoacid dehydrogenase subunit E1 BPSS1711 |
| | | | | succinate dehydrogenase flavoprotein subunit |
| | | | | BPSS1718 |
| | | | | putative AsnC-family transcriptional regulator |
| | | | | BPSS1710 |
| | | | | putative AraC-family transcriptional regulator |
| | | | | BPSS1714 |
| 39 | 2359000, 2367200 | BPSS1720-BPSS1726 | 2 | putative GntR-family transcriptional regulator |
| | (8.2 kb) | | | BPSS1721 |
| | | | | malate dehydrogenase BPSS1722 |
| | | | | putative lyase BPSS1723 |
| | | | | 2-methylcitrate dehydratase BPSS1725 |
| | | | | aconitate hydratase BPSS1726 |
| | | | | (hemagluttinin 78c03 only BPSS1727-BPSS1728) |
| 40 | 2433500, 2448100 | BPSS1774-BPSS1789 | 3 | AMP nucleosidase BPSS1777 |
| | (14.6 kb) | | | homoserine kinase BPSS1779 |
| | | | | putative MarR-family regulatory protein BPSS1781 |
| | | | | organic hydroperoxide resistance protein BPSS1782 |
| | | | | esterase, PHB depolymerase family BPSS1784 |
| | | | | putative molybdenum transport-related, exported |
| | | | | protein BPSS1786 |
| | | | | putative molybdenum transport-related membrane |
| | | | | protein BPSS1787 |
| | | | | putative molybdenum transport-related,ATP-binding |
| | | | | protein BPSS1788 |
| | | | | molybdenum transport protein BPSS1789 |
| 41 | 2467400, 2484900 | BPSS1808-BPSS1826 | 4 | acetaldehyde dehydrogenase BPSS1808 |
| | (17.5 kb) | | | thioesterase BPSS1809 |
| | | | | branched-chain amino acid aminotransferase |
| | | | | BPSS1810 |
| | | | | putative non-ribosomal peptide synthesis thioesterase |
| | | | | BPSS1812 |
| | | | | NRPS related protein/phytanoyl-CoA dioxygenase |
| | | | | (PhyH) family protein/BarB2 BPSS1813 |
| | | | | NRPS related/BarD BPSS1815 |
| | | | | hypothetical protein |
| | | | | putative serine/threonine protein phosphatase |
| | | | | BPSS1819 |
| | | | | AraC family transcriptional regulator BPSS1824 |
| | | | | Glycosyltransferase BPSS1825, BPSS1826 |
| 42 | 2548400, 2563900 | BPSS1876-BPSS1888 | 2 | putative sensor kinase/response regulator fusion |
| | (15.5 kb) | | | protein BPSS1876 |
| | | | | aldehyde dehydrogenase NAD family protein |
| | | | | BPSS1878 |
| | | | | acetolactate synthase BPSS1879 (biofilm formation) |
| | | | | Na+/H+ antiporter BPSS1880 |
| | | | | hypothetical protein |
| | | | | Phospholipase D, putative |
| 43 | 2571200, 2580500 | BPSS1896-BPSS1902 | 3 | 2,4-dienoyl-CoA reductase (NADPH) ‘fadH’ |
| | (9.3 kb) | | | BPSS1898 |
| | | | | putative AraC-family regulatory protein BPSS1899 |
| | | | | putative LysR-family transcriptional regulator |
| | | | | BPSS1900, BPSS1902 |
| 44 | 2589800, 2624000 | BPSS1912-BPSS1939 | 2 | transposase for insertion sequence element IS1001 |
| | (34.2 kb) | | | BPSS1912 |
| | | | | lysine-specific permease BPSS1913 |
| | | | | putative metallo-beta-lactamase family protein |
| | | | | BPSS1915 |
| | | | | zinc-containing alcohol dehydrogenase superfamily |
| | | | | protein BPSS1918 |
| | | | | F0F1 ATP synthase subunit alpha |
| | | | | putative methyl-accepting chemotaxis protein |
| | | | | BPSS1927 |
| | | | | putative outer membrane lipoprotein BPSS1929 |
| | | | | putative ABC transport system, exported protein |
| | | | | BPSS1930 |
| | | | | putative ABC transport system, membrane protein |
| | | | | BPSS1931 |
| | | | | putative TetR-family transcriptional regulator |
| | | | | BPSS1935 |
| | | | | putative outer membrane efflux protein BPSS1936 |
| | | | | putative ABC transport system, exported protein |
| | | | | BPSS1937 |
| | | | | putative ABC transport system, ATP-binding protein |
| | | | | BPSS1938 |
| | | | | ABC transporter Class III Family o228 - Function: |
| | | | | Drug resistance #72 |
| 45 | 2629700, 2638500 | BPSS1942-BPSS1952 | 2 | Alcohol dehydrogenase BPSS1944 |
| | (8.8 kb) | | | ATP synthase gamma chain BPSS1945 |
| | | | | ATP synthase alpha chain BPSS1946 |
| | | | | putative ATP synthase B subunit BPSS1947 |
| | | | | ATP synthase C chain BPSS1948 |
| | | | | putative ATP synthase A chain BPSS1949 |
| | | | | putative ATP synthesis-related protein BPSS1951 |
| | | | | putative ATP synthase epsilon chain BPSS1952 |
| 46 | 2688700, 2705700 | BPSS1988-BPSS1998 | 2 | hypothetical protein |
| | (17 kb) | | | peptidase, family S15 B[SS1992 |
| | | | | serine metalloprotease BPSS1993 |
| | | | | metal-related two-component system, response |
| | | | | regulator BPSS1994 |
| | | | | heavy metal TCS sensor histidine kinase BPSS1995 |
| | | | | class D beta-lactamase BPSS1997 |
| | | | | putative lipoprotein |
| 47 | 2713900, 2725900 | BPSS2009-BPSS2015 | 3 | glucosamine--fructose-6-phosphate aminotransferase |
| | (12 kb) | | | BPSS2009 |
| | | | | outer membrane protein |
| | | | | putative inner membrane glycosyl transferase |
| | | | | BPSS2015 |
| 48 | 2771200, 2794600 | BPSS2053-BPSS2059 | 2 | On GI 16 |
| | (23.4 kb) | | | cell surface protein/hemagluttinin/adhesin/ |
| | | | | hemolysin BPSS2053 |
| | | | | YD repeat/RHS repeat protein BPSS2054 |
| | | | | Rhs element Vgr protein BPSS2056 |
| | | | | putative ATP-binding inner membrane transport |
| | | | | protein BPSS2058 |
| 49 | 2802200, 2819100 | BPSS2067-BPSS2081 | 3 | On GI 16 |
| | (16.9 kb) | | | aldose 1-epimerase BPSS2067 |
| | | | | ABC transporter, ATP-binding protein BPSS2069 |
| | | | | ABC transporter, substrate-binding protein |
| | | | | mandelate racemase/muconate lactonizing enzyme |
| | | | | BPSS2072 |
| | | | | GntR family regulator protein BPSS2073 |
| | | | | senescence marker protein-30 BPSS2074 |
| | | | | transposase, IS66 familly BPSS2076 |
| | | | | putative DNA-binding protein BPSS2079 |
| | | | | hypothetical protein |
| | | | | alpha-galactosidase BPSS2081 |
| | | | | putative ABC transporter permease BPSS2082 |
| | | | | ABC transporter Class III Familiy MOS # 61 |
| | | | | BPSS2069-BPSS2071 |
| 50 | 2946500, 2970900 | BPSS2179-BPSS2022 | 2 | hypothetical proteins |
| | (24.4 kb) | | | putative glycosyltransferase BPSS2182 |
| | | | | putative pilus assembly-related outer membrane |
| | | | | protein BPSS2187 |
| | | | | putative pilus assembly-related, exported |
| | | | | protein BPSS2189 |
| | | | | putative pilus assembly-related protein BPSS2195 |
| | | | | type II/IV secretion system-related protein BPSS2196 |
| | | | | putative AsnC-family regulatory protein BPSS2199 |
| | | | | aromatic amino acid aminotransferase BPSS2200 |
| | | | | porin related, membrane protein BPSS2202 |
| | | | | Putative tad-type pilus (complete) |
| 51 | 3032400, 3049000 | BPSS2257-BPSS2269 | 2 | Fusion protein, ATP-binding transmembrane ABC |
| | (16.6 kb) | | | transporter and regulatory protein BPSS2259 |
| | | | | phosphatidylserine decarboxylase BPSS2261 |
| | | | | 4′-phosphopantetheinyl transferase superfamily |
| | | | | protein BPSS2266 |
| | | | | ABC transporter Class I Familiy DPL # 15 |
| | | | | BPSS2259 |
| 52 | 3069700, 3098800 | BPSS2283-BPSS2302 | 4 | Sulfate transporter family protein BPSS2285 |
| | (29.1 kb) | | | HSP20/alpha crystallin family protein BPSS2288 |
| | | | | Putative porin protein BPSS2289 |
| | | | | AMP-binding enzyme BPSS2290 |
| | | | | GerE family regulatory protein BPSS2291 |
| | | | | taurine catabolism dioxygenase TauD, TfdA family |
| | | | | protein BPSS2294 |
| | | | | putative transport protein BPSS2296 |
| | | | | molybdopterin oxidoreductase family protein |
| | | | | BPSS2299 |
| | | | | iron-sulfur cluster protein BPSS2300 |
| 53 | 3125100, 3150900 | BPSS2323-BPSS2334 | 2 | hypothetical protein |
| | (25.8 kb) | | | Putative permease BPSS2324 |
| | | | | ABC-transporter ATP binding protein BPSS2325 |
| | | | | beta keto-acyl synthase/PKS BPSS2328 |
| | | | | RND efflux transporter |
| | | | | putative acyl transferase BPSS2329 |
| | | | | putative aminotransferase BPSS2333 |
| | | | | Probable PKS/NRPS cluster |
| 54 | 3158500, 3166100 | BPSS2342-BPSS2348 | 3 | GGDEF response regulator protein BPSS2342 |
| | (7.6 kb) | | | sensor histidine kinase/response regulator BPSS2345, |
| | | | | BPSS2344 |
| | | | | NADPH-dependent FMN reductase domain protein |
| | | | | putative arsenate reductase BPSS2348 |

1. A method of selecting an anti-macrophage micro-organism comprising an anti-macrophage factor, the method comprising the steps of:
a) obtaining an assay micro-organism and preparing a sample thereof;
b) lysing the assay micro-organism cells contained in the sample from (a) to form a lysate fluid;
c) contacting a sample of macrophage cells with the lysate fluid from step (b);
d) determining the macrophage cell viability and comparing the viability to the viability of macrophage cells in a control macrophage sample; and
e) selecting the micro-organism as an anti-macrophage micro-organism if the viability is reduced by at least 10%.

2. The method of claim 1 wherein the step of obtaining an assay micro-organism comprises introducing genomic DNA from a test micro-organism into an expression micro-organism and using the expression micro-organism as the assay micro-organism.

3. A method of identifying a gene encoding an anti-macrophage factor comprising the method of claim 2, the method further comprising the additional steps of:
(aa) before step (a), preparing a genomic library of the test micro-organism genome in an expression micro-organism and obtaining expression micro-organism clones, each of which comprises an assay micro-organism; and
(f) after step (e), determining the nucleotide sequence of the test micro-organism nucleic acid in the selected assay micro-organism; and
(g) using the nucleotide sequence to identify an equivalent sequence in a test micro-organism gene and selecting the gene as encoding an anti-macrophage factor.

4. The method of claim 3 wherein steps (a)-(f) are repeated with an assay micro-organism from at least two clones obtained in step (aa) and replacing step (g) with the steps of:
(fa) comparing the test micro-organism nucleotide sequence from each selected assay micro-organism with the sequence obtained from a different selected assay micro-organism to identify one or more regions of sequence overlap; and
(fb) using the nucleotide sequence in each region of overlap to identify an equivalent sequence in a test micro-organism gene and selecting the gene as encoding an anti-macrophage factor.

5. A method of obtaining an isolated anti-macrophage factor, the method comprising the method of claim 3 and further comprising expressing and isolating a protein encoded by the gene selected in step (g).

6. A method of obtaining an isolated anti-macrophage factor, the method comprising the method of claim 4 and further comprising expressing and isolating a protein encoded by the gene selected in step (fb)

7. A method of determining whether a test compound is an anti-macrophage factor comprising introducing the compound into a micro-organism to form an assay micro-organism and carrying out the method of claim 1, wherein the test compound is identified as an anti-macrophage factor if the assay micro-organism is selected in step (e) as an anti-macrophage micro-organism.

8. An anti-macrophage factor obtained using the method of claim 5.

9. An anti-macrophage factor obtained using the method of claim 6.

10. An anti-macrophage factor obtained using the method of claim 7.

11. A vaccine composition comprising an attenuated or inactivated version of the anti-macrophage factor according to claim 8 and a pharmaceutically acceptable carrier.

12. A vaccine composition comprising an attenuated or inactivated version of the anti-macrophage factor according to claim 9 and a pharmaceutically acceptable carrier.

13. A vaccine composition comprising an attenuated or inactivated version of the anti-macrophage factor according to claim 10 and a pharmaceutically acceptable carrier.

14. A method of treatment or prevention of a disorder caused by an anti-macrophage micro-organism comprising obtaining an anti-macrophage factor using the method of claim 5, and administering a therapeutically or prophylactically effective amount of an attenuated or inactivated version of the factor to a subject in need thereof.

15. A method of treatment or prevention of a disorder caused by an anti-macrophage micro-organism comprising administering a therapeutically or prophylactically effective amount of a vaccine composition according to claim 11 to a subject in need thereof.

16. A method of treatment or prevention of a disorder caused by an anti-macrophage micro-organism comprising obtaining an anti-macrophage factor using the method of claim 6, and administering a therapeutically or prophylactically effective amount of an attenuated or inactivated version of the factor to a subject in need thereof.

17. A method of treatment or prevention of a disorder caused by an anti-macrophage micro-organism comprising administering a therapeutically or prophylactically effective amount of a vaccine composition according to claim 12 to a subject in need thereof.

18. A method of treatment or prevention of a disorder caused by an anti-macrophage micro-organism comprising obtaining an anti-macrophage factor using the method of claim 7, and administering a therapeutically or prophylactically effective amount of an attenuated or inactivated version of the factor to a subject in need thereof.

19. A method of treatment or prevention of a disorder caused by an anti-macrophage micro-organism comprising administering a therapeutically or prophylactically effective amount of a vaccine composition according to claim 13 to a subject in need thereof.

20. An anti-macrophage factor comprising an amino acid sequence comprising one or more of SEQ ID NOs:1-9.

21. A vaccine composition comprising an attenuated or inactivated version of the anti-macrophage factor according to claim 20 and a pharmaceutically acceptable carrier.

22. A polynucleotide encoding one or more of the amino acid sequences SEQ ID NOs:1-9.

23. A vaccine composition comprising a polynucleotide according to claim 22 and a pharmaceutically acceptable carrier.

24. A method of treatment or prevention of melioidosis comprising administering a therapeutically or prophylactically effective amount of an attenuated or inactivated version of the factor of claim 20.


SEQUENCE LISTING (9 sequences)

SEQ ID NO: 1 - protein, 2031 aa, Burkholderia pseudomallei
MAKTSGTADEILNLPSGGGSVSGDGGDFSVDLNTGTATLKFDLTVPAGPNGITPPHTLQYSAGAGDGAFGIGWSLGLMTIRRRITPATGAAEPAPPGACSLVGVGELVDMGARRFRPIVDATGLLIEFTGASWTATDKTDTQYTLGTSANARIGGGALPAAWLVDRCADSAGNAIAYTWLDVGGARVPQAIAWGTYRLDFVYEARPDVLVDGSYGAPVTLDKRCARIELHATTEAPSLVRSWTLLYDDDGGRGRSLLATIREQGHAADGSILAAPERTFAYSSPGAPALVPVTGWTTPLSDPNTDLVDLNGDGLPDIVHLGHGMPTMHPNLGGGQFGAPRPLARTAAPLRLSAPSVAFADMSGNGSVDVLVLDAPFSGYYPLAAPGGSAPAGFGRPIVFERAPAVSPSDVRLRFVDLNGDGITDILLDTGRGWLAYLRDGPASWSDAPRVLPPARTPPVSLADPHVHLADMTGDGLMDIVRVTGGGLTYWPARADGGWDAAIAMTPSPAFGRDFDPHRLALYDVDGDGCADLVYVGLQSVTLWRNVGATRLAAPVAIPGTPVALPGSYRIVDLLGSGTAGVHFQLPSIAGASRQSYLDLGGGVKPYLLTDLAHGAAQSTHIGYRTSTEYARDDAQAGAPWRTYHPFPIQCVARTDQTDHGTGATTSTRYAYHDARYDPATRTFLGFGRVDSEQLGDASCPTLRVETTFHLGLDPADPARPLTGDEALKLGALRRKPLVTATYGLDGSPLEHRPYSITRHAYDALLVASTADNGKRIAVPYCTTSTEERWERQSAAVSARTVDYLAITSEGDVTSQRTRAQRAGIAAPDQDVTTTTTLATGGKNLRLPARVTQTAPGGDIVSESICFYDGDAFAGLPEGQATRGLVTRIEDRVFDDAFVASVWGDTPPDLTQYGYHRLPGEAGSWWKTRRAHARGANASGPTLATKGPLGALQTLQYDASGQRVVKVIDALGNAVAATTDARVFQTASLTDANGHRTADAFDALGRVVATIGPLDTPALPTIAFTYTVGAISTVTSATRGTHGGADAVPALTWIDGAGNVLGKGTPAAAPGEWTVTHAVRRNARGLVAASFLPYAATGANWQPPPADTGATTSTYDALGRLVQLTRPDGLVVTSRREGDTLITSETWPGGAALDVERQVYDAAGQLVSVSRNAGDHWVEQRYAYAPSGKVRDVTLPGGAHVAFTLDLLGRVFAQQSPDTGRTVFLLDASDNQRARTNAAGQIVRTEVDAMNRATNVYHDAEPAPRVRYEYADASGAPPADGIVANRYTRLWRITDEIGTVDFEYDEAGRKTATTRTVAATGQRFVTRRAYDALGRLARATLPASAPGGAARTIAYGYAADGRLVSASGVVKDAAYDRFGRLTSIDYENGASTLIDYAANAGGIARVRVLDAARNVLRDTTLTRGGGLVQTLASAHAGDDSVDFGYDPLRRLTSAHYRQGATAADAHDWTFDDAFNATAATDAGVLTYEPGTHRLASVGGAAVAFDVAGRMTSGRFGAATFDAADHLSAVTLPDATHIAHTYDYQGRRVRSTKNGAQTYFSPIEDIEFQGDTAIVWITFGKQRIAADIGGALTFVHPNALGVMDLITDSGGAYGTRVRQTPFGYARAADGAAPAGGVAAVALALGAADATGLVCHGLRWYDPCVGQFISPDPVVTSVYTVGAWNPYVYCLGNPILLADPNGCSFLSVLEIIGVAILAAACVVGAIFTGGATLVALGVLSANIGGWLLAGVALGSLGGAIAGELAAQKAGGNLWAGAFLGAFLGGATSLIGGALGGAAAAGIDTLIGGTKTFLSFVAAGAIQGTLAGAGTGLAIGYAGGKGNAESMLIAMAKGAAWGAVLGTLLGAGIGAIAGTGISGAAKPDNFLNIGAFGQKFADFTSTSTAINSADNAAGVTESLAQLSMPNGFNAGNLLGLLPNLVTTNEQAAGWFSIPIGWLGPGVLNDAGFAGLVDTSMALDQAGFSYAHQISLLLGAAPYFIDYAATMAQIVDANGVNNFEMAFNNAFGSGSPSNTG

SEQ ID NO: 2 - protein, 1094 aa, Burkholderia pseudomallei
MPGGATMLSLTQRVLTYSFIDRPPVNPRAIGTSSADAALLAQIDALLASAAASFKARAYDAALDDYFACESLIYSHLDAQWNPDLGGRLRSRLPRDAALFDSLLSATSQWLNVLPVPAPASPVRPATPPPAQALAGVAALRGAGLAPVSPNPAATAQALSDMQLASLYTSQGNSAASSVAVTRAKAVDAAVVGAFSPPQMPNPSALPAANPNAAPSTTPGFHPAPGVMPPRGIDLAPAALTPLKIQPMPKLPIALLAQKQVGLLTGSGAQTAVKAIQWAASGAPDIASIKTILYAPHASAAALPDALTNANSLWERSVLLPHDYFYTIPLAIAHCYQALGDYANAETYYLQAAGYAYLNTATEGPYIWVALAQLYRAWGDSLYLQGDRAGATNAYGKVVTPGSPAAPATALYQLAGLATAAKRATALLPQLATLAQTGTGGVTADDVAIATVLLEVYAKLVQIGAGLDYWGNYAAAVPIWSFSYLQQVAINFAQLAQQAENQVVNFWNQADQAKLTRTELANQVSQASGQINAAQQQLAVAQAQAQAYQAGVALAQTRATNAAKNAQEYGSLNSQVIVIQATGQQVSGGDDGDYNGVSAMANQYLSGQRISGDSATVAAATNLAANRLSQQFQIDSMNRTTAEMQQALAQAQAQLAAANAQVSAAGANLAVAQLNAQAAAQTLGVFDADTFTPQVWKAMGNFVDQIYERYMNMALRAAKLMQQAYNFENDVSVSFIKASYQGVVDGLLAADALMADIQSFTDDLVNAKRGKKQYLKQSISLASRYGYLFETQLRKTGTMTFETTLDDFDSAYPGTYQGRIRRVLVSVQGIVPPTGISGTLGNEGISFYRLPADVATPAAPSKVRVQSAETQVISDYDPVQDAVLAPPPENQTGIFEGAGVASSWTLSLPPALNDINYGTLTDVVLTFLYEARFDPRLVQPVLAQLASRPGFYNRERAIPLAWLYPDLFYGFVSTGTLTLNLSAADFPIDQTAPAVTAVSLLVAMKPGTPASNVTIALAAPGKGALSGVTDATGAISSQSAGSAWAGAVGGAALGDWTLTLGAAANPSLAPGGKLDLSPLINLVLVIDYAFKPRG

SEQ ID NO: 3 - protein, 728 aa, Burkholderia pseudomallei
MTTPYPSPPDARANRAAEARAHGGLLEPGRNCDALLHAERFATLIDGDAYFSTLRAALRRARHTVFIVGWDIDSRLRLVPDGAHDGWPEPLAAFLHALANARRRLRIYVLAWDFSMIYALEREWPPVYRTAWRSRHGIVFRLDGTHPRGASHHQKFVVIDDRVAFVGGFDLTRSRWDTPRHAPDEPRRVDPNGARYAPFHDVQAMFDGDAAAAIGALARERWARTCGKRVAIRAHRRRAADDDPWPPGAPVDVRDVTLAIALTTPRRRGGDEVGHIRALTADAIAAARRDLYIENQYFTAAVVREALTARLAADDPPDVVVVAPREQSGWLQEATMGVLRARLHRALVDADRHGRYRLLCPHVDGLGELCVNVHSKLMCVDDDYLVIGSANLNNRSMVLDTECNIVLAANGEPRVREAIARTRERLLAEHLGVSADAVAAARACTGRLNRAIDALRGAHPHRTLRPFTPSASADLDALVPVSAWLDPERPIDADRLMSEFVPREQSRSLTARFLALGAFVLFAAALAAAWRFTPLGQHLSVASLARAGARAAELPAAPVALVAGYVAAALLAVPVTLLITATGLVFGAWPGIAYAAAGTLLAAAATYGIGRWLGRDAVRRLAGRRANRLSEHIGRRGIVAMAVLRLLPIAPFTVVNLVAGASSIGFRDYLVGTALGMLPGIVLTVTFAHQLSAALVHPTPAAFAWLAAIGLAFVGVSALLLRALGRRR

SEQ ID NO: 4 - protein, 908 aa, Burkholderia pseudomallei
MNRNVFRLVLNRVAGMPVPMPAAEVSRGRGKLGCGGVRAQRRGGAACAALLGVAGPSLAFAAVVADPNGGAQRPGMATTANGTDLVNIVAPDATGLSHNKFNEFSPVGRGVVLNNSVRPGESQIGGMAAQNPNLMQPATRALLEVTQQRSVLQGTLEAFGGKLDVLVANQHGVTINGLTTLNVGRLGVTTGQVLPQAAGQLRLGVTQGDVLIDHGGIDTQGLDMFDVVSRSIAVRGPIHDSSRAAGADVRLVAGATAYDPQTGHYEAIAADESKAPVQEGISGELLGAMHGRHIVLVSTESGVGVRHDGPIKSANDIRVSANGEVTLGGPQQAAQEAVAGAQAVGGAGMQNVIAGGTVSVCARGHVAIQGAVTAGQDVDLQGKSVKAGRMSAQRDALVTAADGVTLDGPVDAKRHVWIGAHGDVVIREAAAEQNVVLLGRSVTAGRLDAQRDVLAAARDGVTIHEAAAAGQDVVLQGSSARVGQTSAQRDVLVMAADGVTLDGPVSAQRAVWVETQGDVAGSEWIKAGRDVQIGAAADLAGAVTAEEMQQLKAHGDAANRRRVKAGRNEPAGTAAERPAAAEQTVAVADAMREIGVGGDRLSGLDAAPGTPGTPFGAHPQAMFDDPAAQIARSARSTATAGGHAGSFMRVGDGHIAKMTTSREAEIYENYRLALAGVIPDTVPPEEVDSRVGVTARQRQAMATFKGWAEMKGQRVVVMQALGAEIAPEDKIELDVKIGASTVSRTELIGAGRTRWQALSKKVRLTAADLLRGSRSLVGDDRGYTLAGRTSGGIALDARNSRNSVGRSSESLIREALDRSPDTRWRNAQHLLGQLQTIREKMHALPLTFVASSVLIAIDKRKPENSVARLIDLAHPVQPFENEADYEKVNHRFEDGLDKLIRLFQQVEK

SEQ ID NO: 5 - protein, 563 aa, Burkholderia pseudomallei
MTHIVLTLAMTVLLAGRSAPASAFGPTVQDRERDLGEKARRQQMIEQHRRGHAAEPAAGLPAPVQAPELGGARFPITQIEVRGGARHSREIDRIVERYRGATMGQAEIFALLRDLMNFYYGRGYITTGVSLAEQNLSSGKLIVVVHWGYVKGWRVNGKAPEGMRERLMLSSAMPGIEGSVLNIHDIDQAIENLNGLGRAAKINVVPAEEAGYSYLDVVLQRSKPFAVSASVDNSGLGDRAGDGLYRYGVDIAVAHLLGWNDVLTLSAGRRYYQDPAHHAYDSLAVSYGWRVGRWSADLRYIAQPTKSLLSGTYGDYQLSGNTQDVNLRVSRVLRRWRTGTDAVYVAIDRKRVRNYIDDTLIEINSNTHTSVTAGVNRLDSLFGGVVFADGGWTRGVGWLGASEDPVSAGQGPLSRYDKFNLNLNWSRDFTAGSLRARYTAAMGAQYSKDDLYYDSKFVIGDQYTVRGFKIRSAYGDSGVSVSNTLSFPFPTSIGTVTPLVGVDWGAVGNNGEADNGGWIGAVAGGLRVSTKWANAAVTIGKPFRALPGMGYGVVWYLSASATY

SEQ ID NO: 6 - protein, 1071 aa, Burkholderia pseudomallei
MNMRDTSLRPLLPAQREIWLAEQLRPGTGVYNTAGYADIVGAVDHGALRDAFGHAMREADAARATFSAHGDDAWQTPRDAPHADVPLVDVSGQADPAGVALAWMMRDMRTPPDFAAGPLVRSALLRVGAARFFWYVCAHHIVTDAYGTALVVQRTAQLYGARVRGAAPPPAWFGTLDALIDEDRAYRESAAFADDRDYWRARLAAAPEPRSLSPVALPGQFAPAGDFHRQWGELDAATSDGLRAMARAGAQGMPPLVAAMMAAYVHRFTHERDVVLGLPVMARLSRATRRTPGMVANVLPLRFAFDRGTTFAALHAQCAAEMRASLRHQRYRGEAMLRDAQQRRPAERLHAQNVNVMAFERALAFGDCPARSHSLSNGPVDDFSITVYDDGARQPIRIAFDANAARYASGDLAAHRERFLRLGVALANAAQRPIADAQWLTPAERRTLVGERGTPAPDAGEPFDTIAARFAARAAERPDAIALVDRGRRITYGELNARANRLAHVLIEAGVGPEALVGLHMPRSAELVVGMLAILKAGGAYVPLDPAYPASRIEFMVADARPMLSITTGEHAAQLPARTPTIVLDAADAQAALRRAPAHDPVRPAPLDREHAAYVIYTSGSTGKPKGAVVSHRNVIRLLDGTRGWFDFGAAHTWTLFHSFAFDFSVWECWGALLTGGRLVVVPYDVSRSPAEFLKLLVDERVTVLNQTPSAFRQLMQADEAHADLSARLALRYVVFGGEALDARSLARWYERHADTAPRLVNMYGITETTVHVSYLALSRAIAGMPANSLIGRPLPDLRVYVLDAALRPVPAGVPGEMYVAGAGLARGYLRRPSLTAQRFIADPFGPPGTRMYRTGDVARWRADGGLDFIGRADEQVKVRGFRVELGEIAARLACDPSVAQAQAVVRQDGPAHERLVAYVVPRAGATIDVCALRASLAAEMPEYMVPAAIVALDAMPLTPNGKLDRAALPAPIVTGTSRRAPENRIEQQVCAMFAELLDAQTLGAEDNFFELGGDSLLAMRAINKLRQTFDVELTIRDLFSAPTVAALSMRLDAQLAARRAHADGAELPAG

SEQ ID NO: 7 - protein, 68 aa, Burkholderia pseudomallei
MTHLLDDPDGSFQVLVNEEGQHSLWPAELPAPAGWRRTFGEAGRQACLDHIEAEWTDLRPRSVAARCA

SEQ ID NO: 8 - protein, 429 aa, Burkholderia pseudomallei
MDLHERTLDAPGAPAAAAHGAGSRLSREVWIILLTLAVDAVGLGVATPVLPDLLRAVGERPAHVPVMLGSLITCFYFMQFVFGPIVGALSDAWGRRPILLIALGGGALSYAIGATARSYGGLLAAHMLAGIAASSAAVATAYLADVTPPALRARRFALASSVLGLGLIAGPAFGGVLGALGPRVPFVAAGAIAALNFVSAACFLRESLPARRRVPVSWARANLFGSLRLVREDRVFRDLLAAVCLGMLAYGIYLSCFVLANAARLGWGPRENGLALAGLGLGITLTQTLLLPRLVARLGEHRTALVGFGAFVLAYGLYSRADSVALVAAALAVHALSLISDPSVRSLISVHAGPQRQGEYQGALVCLTGLASSIAPLVGGNLFAFFTRAGASTYFPGAPFLAAAVLYALAMLAVWRAMAGAARRRSASH

SEQ ID NO: 9 - protein, 4236 aa, Burkholderia pseudomallei
MNHRLPHVERGDADEPSIETAAGEPPPLALPASVAQRRLWFVENGDVRASTYNVPAAFTLTGPLDDAVLERALAFMQQRHPALRSRFRTRDGELRIELAPQPAPLARQDLGALDADVRARTAERLCANHANRRFDLERDAPIRCLLLRLGENEHVLAVNVHHIVFDDWSIRIFFRELGAVYGALLAGATPDLPALDYAAAVAASVPAAARHAAARQYWARAMSGAPTLHKLPTDRPRPAAPRMRGAVHKHVFARRHAEGIRALCRRAGVTPYMLGVAAFAALLHRYSGEDEIVIGSPFANRVTQAQQSLIGFFINLIPLRVRFDAGVNFLDLLAQVRETSFDAFEHAVLPFDQIVDAIRPPRSSSHAPVFQIMFDYLKSGGMLELDGVGVTGSLVHTGTAKYDLTVSMEEGPDELAAIVEYDTDLFDAGTIARLGGHFERLLENVLASPAAPIAEGSLLPADELRQVRRFTRPDEPYAHIPFSPMPQRIREAARRAPHAVAIVHGDARMTYETLDRRSDALARALRARGVGRGSRVASLQSYSEKIVVAYLGILKAGAAYLPLDPADPRRLEKIEDAAPAMIVTARRDLEDVPQALRARTLTIDDPIECGKAPDAVNDVVTDVATDVVTDATARDAELDFATLAEADPAYVIYTSGSTGKPKGVEVSHGSLNVSYHGWHRAYRFGKPGHPVTLQLAGMTFDLGIGDVSRTLACGGTLVMPPRDGLLDAGRLHALMRAERVSFGDFPPVILRELIRHCNETGDRLDMLDTLVCGADVWFGHELHAARALCGPHARVLGSYGVTEAAIDSSYFDPDLHALAPDSVVPLGRPLPSCELLIVDPLLQMTPIGVPGELLVAGPAVATRYLNNDALTAQKFLRGRVDEHGRVIAGDGQTRFYRTGDICRFLEDGTIDFLGRRDNQIKIRGFRVELGEVEGVLAAHPDVRQCAVVVRDEASGDPSLAAFVVSDAPIAALRGYLRGRLPAYMLPAAIERLGDMPLTASGKIDRNRLKAWPLSAPDVPPPDAATDVERRLLALWENLLSARVPSVHENFFQCGGHSLAAARLASSISQAFDISIGVSSVFNHPSVAEQARLVEALASARAPRDVRQTAHADAAEPAGDDGLLSYSQQSLWLTAKRTPDDFSYNIPVTWRLDGPLDAHALERAINDVVARHDALRTVFSSDVRTVVGPHRESSQEPTQRVLDTLTIALRRVAVAPDDAASLPARLREAHSRAFDLNAGPLLRAVLFEIAPTHHVLDVTIHHIVIDGPSFGLFWRDLQTAYRARVAGEAPGWQRPARRHADFVSRQRQALRGEAAARQLAYWREQLRGLPAALPLPDAVLAAHAPGSARSLTFEMPDDVAAGLAALARRTNGSPFIVYLALFAAALRQQTGEADFAIGTPLSLRPHEGFADVLGFFANTMPLRMRLHGLDTFERVLRYVREQCLALYENGDVPFEYLVQALKPARAARRNPVFQTIFSCEFDDERLQLTGVDAHALALDAYTAKLDLEMAINVSGGRVVCHLMSRPGSFDADALSSIRHHFLRTACSATRAHAEREAAEARATHEVHEAHEVHEVHEVHEMHEMHDVHEARAQLADDARGARRPSADDLFGLFARSAARHAQRVALDSPTLRASYAQLAERVSAAARALAAHGVRRGDRVGIFVGHHPHNVTAMLAIARVGAAFVPMDPEHKPQWNRHIVDDAALTALVGGAWTADAARGFGLPVVDLDAPPPPASELADAPAAGGAHPDDCAYVIYTSGSTGRPKGVAVSHASVCHNVRAMAEIMRIGPQSRMAQYVSPVFDVVLGEIFPALAAGAAIVFAERRRPLPGQALVDWLDAQRVSHVWIVPSALAMVPEAALPALEVLIVAGEACPRELAQRWAAGRRLLNGYGPTEAAIVVSLTDYHAQRERLILRPMGGARLHVLDEALRPAPAGAAGELFIGGACVAQGYLGQPARTAQAFVADPFDAEPGARMYRTGDVVRRLDDGAIQFIGRVDRQVKIRGFRIELDAVRAALMEVPGVQAAEALAQPDASGQPLLVGYVVARRAKAELLDALRGKVPDAMVPSTLVFLDALPTGSTGKTDLKALKALKTGDAARPAAAAADMPRAASQGRTLHRVREIWRTLLERDDIGDDENFFDAGGHSLRAVALHQRITEAFGDVIALTDLFEHPTIGALAAHLDAFAPRDGEAADDAAGAAARAPADGVLDTDAIAVIGLAGRFPDAPDLDRFWERLLAGYEAGRTLSDAELDAHGVPAELYRNPHFVRRFKELEGKAEFDAGFFGYSPREAQVMDPQQRIFLELAWQALEQAGYGDRGRVRSVGVFASAAFNYYLVQNVMPNAERLRLEPGQWLIGNDKDFIATRTAYKLNLLGPALSVGTACSSSLMAVHLACASLRNGEAQMALAGAVALDPDQVGYLYAEGGIMSPDGRCRPFDAAAAGTAGGSGGGVVLLKRLDAALRDGDTVYAVIKGSAANNDGADKVSYTAPSVAGQTAVIRDALRAARVSADSIGYVEAHGTGTPLGDPIEVRALAQAFAEAAAPGALANGRCGIGSIKGNIGHLDAAAGIAGFIKAVLALHREAIPPSINCETPNARIGFDKTPFSVVREARAWPRTATPRRAGVSSFGVGGTNVHVVLEEAPRVRAGESAEPSRWQLLPVSARSPSALREQWRQLRDALAHARPRVQDVAHTLQVGRTAFEHRGFAVVDAAADAPAQLDAAGSPPAFERRAAPPVVFMFPGQGSQYPGMGAALYRSGGVFQAEVDRCAQLLRAHLDRDVRSLMFDAGASLLRETRYTQPALFAIEYALARQWLAWGVTPHAMIGHSVGELVAAAVGETLALPDALALVVARADAMQRQPPGAMLAVLADARELAALNGPGCEIAAINGPEQYVLAGDAARIAALEDACIAAGVACQRLATSHAFHSSAMDGAAREIDRASERIVRRAARIPLISNRSGRWLNEQDLRDAGYWGEHVRQPVQFHAGVRTLLDALDAPIFVEVGPGRALGNLIGGWAGLGPQRIVATLPHARERRDDMAAALRGVGTLWAQGVDVNWAGLHAPGAARRIALPTYPFERTRHWIERPAGARAAPAREADGVPMRRGEDAADGSLTVSFALHERLWFLDEHRIFDGAPVLPGTACIELVRRAYSLVRPGAAMTMRDVYFPTALILSTDESRNVRVVFRPPERVSDGAAAGGDLAFVLESNDANGPAGWTPHASGRIGDDPPACAAPASLAALAALDAPAALREQWGLTALDDVAALSAQAFADYGARWRGVDALWLGERAGLARLRLPAAGSGDLPDFALHPAMLDVATAFLPACLRPRDASVPFRYESIRMHWPLRADCYSFAVETAPNVYDVTLFAWDEAARRADVLVAIGGFARREPAHRARDVAQWCRTVSWRDAPAARALPPERWLVFGDEWFALAPAGSVLVREDDAFRAHGDNGYGVRPGEKADCDRLIARLAEQGGVPAHVVYGWAQTDVDRAFAGLAALLQALGAHPADFRVSLVTKGARSARTLDACAAAAPAGLLKAVRWEYPRIVCRHIDIDDASDATIDALRAELSSEPATPPGAPPELPSSIALAGARREAPGFAALPAVARDDVLRDGGAYLITGGASGIGLELAAHIASRRRDVRLALLSRSPHDENAARFAALDEAAASVLRLTADVAHAAQLADALRTVRARFGRIDGVIHAAGVEASGLLETGTPDAWRRVMAAKVHGARHLFDQLAGDPPDFIVLCSSLAAVVGGLGQADYAAANGYMDALAQHWRQRGVAAIAIDWDTWSDTGMAFDHAARTRRSNDRPGALPGLANREGRALFDLALAHDASRIVISKRGFEQDRRDAPTRARRAAAPGDAQAALVALWQELLGVEQVGVDDDFFDLGGHSLLATQLISRVRDQYARSPTLGEFLEEPTIARILRAIDHTGGDTAGDMSGDTAGDAPDVDETLRYCVVPMVKAGSGAPFFCIPGMGGNITQLLPLANALGADRPVIGLQYLGLDGKHAPHASVEAIAAHYVRCIRSVQPAGPYFLGGHSLGGKIAYEVARRLHAQGDAIGLVAMFDSAAPPYSFVAHQDDFAIASMILGVFAYYAGKMEMMAGIDDARLRDAPRERLLAFMGERLAQFGVIQSQSDTSAIRGLFNVYRAAADFSARYAPPHEHLPLPILLVKATEPMPDGIKLPEIRETPAWGWENFTRLPVRTCEVAGNHYSCLMDGYVERIADALRDALASARQAIEA